(19) United States
(12) Patent Application Publication
(10) Pub. No.: US 2007/0088230 A1
(43) Pub. Date: Apr. 19, 2007

(54) Medical instrument and medical equipment for treatment, and rotational handle device

(75) Inventors: Tsuyoshi Terashi, Tokyo (JP); Seiji Shimura, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: FMD CO., LTD

(21) Appl. No.: 11/515,838

(22) Filed: Sep. 6, 2006

(30) Foreign Application Priority Data
Sep. 6, 2005 (JP) 2005-257687
Jan. 17, 2006 (JP) 2006-008161

(51) Int. Cl. A61M 25/00

(52) U.S. Cl. 600/585

(57) ABSTRACT

A rotational handle device rotates a medical instrument for vascular treatment in a tubular form including a tube for insertion by guiding of a medical guide wire inside. The tube includes a helical coil of metal, a tubular tip of metal secured to an end of the helical coil, and a cutting head of a blade shape, formed on the tubular tip, for cutting a lesion upon being rotated. A first rotatable tube rotates with the tube, and has a wire lumen. An externally operable second rotatable tube is secured to the first rotatable tube in an axial direction. An over torque preventing mechanism is disposed between the first and second rotatable tubes, and causes the second rotatable tube to rotate free from the first rotatable tube upon application of torque higher than the predetermined level to the second rotatable tube.

### BACKGROUND OF THE INVENTION

1. Field of the Invention

The present invention relates to a medical instrument and medical equipment for treatment, and a rotational handle device. More particularly, the present invention relates to a medical instrument and medical equipment for treatment, and a rotational handle device, in which rotation can be transmitted to a distal end or tubular tip by preventing application of excessive torque.

2. Description Related to the Prior Art

A catheter is a tube shaped rotational medical instrument by insertion in a blood vessel of a patient. For use of the catheter, at first a guide wire is inserted the blood vessel before the catheter in a tubular shape is inserted by following the wire surface of the guide wire. The guide wire inserted in the blood vessel is used for inserting the catheter for injecting X-ray contrast agent, or for inserting a balloon catheter or stent for deployment against a constriction. Any of the guide wire and the tube shaped medical instrument is a composite element having a helical coil disposed on a distal end, and a tubular tip secured to the distal end and supported flexibly.

In the field of the tube shaped medical instrument, JP-A 63-262160 discloses a composite catheter including the catheter and a catheter sheath. The catheter has a helical projection at the distal end. The catheter sheath has a cutting edge at the distal end. The catheter is introduced to the constriction by rotation. The cutting edge of the catheter sheath cuts and excises the constriction. JP-U 5-063554 discloses a structure of a multi layer tube or tubular wire, disposed about the guide wire, and having the distal end in a diameter decreasing form. The tube shaped medical instrument without the balloon catheter can deploy the inside of the constriction for diameter increasing treatment.

JP-A 6-047094 discloses a type of the catheter for the blood vessel, in which a plurality of projections or recesses are formed on an outer or inner surface for dispersing stress of bending to removing folds, and in which flexibility and resistance to twisting can be high. U.S. Pat. No. 6,348,041 (corresponding to JP-A 2002-539901) discloses a guide wire for treatment, such as atherectomy in the blood vessel, having a helical coil of a multi filament type in which plural wires are wound helically, and an end part cap secured to the distal end of the helical coil.

A total occlusion in widely observed cases has two ends in a cup shape. A first end on a nearer side has two end points with a great thickness, so that a true lumen can be reached easily. A difficulty lies in that the total occlusion cannot be penetrated readily. A second end on a farther side has a portion with high hardness and small thickness. However, a middle point of the second end protrudes convexly, so that a cutting edge is likely to offset from the middle. This problem is serious in particular if calcified content with high hardness is contained in a position short of the second end. A direction of advance of the rotational medical instrument is likely to alter by following a softer portion in atheroma or tissues. False lumens may be reached and also penetration of a blood vessel may occur.

Also, a medical tube is known and available in the market, and has the tubular tip at the distal end. There is a problem in a lag of rotation, namely in that transmission of rotation upon applying torque to a handle device of the tube shaped medical instrument is delayed at the distal end. It is likely accidentally to transmit excessive torque to the distal end, to cause perforation of the blood vessel or other false treatment as failure of the tube shaped medical instrument.

The medical tube having the tubular tip being cylindrical at the distal end is simply cylindrical of which shapes of sections of the distal end are equal to or similar to one another. There is no perforating structure. If the hard portion of the first end on a nearer side is thick, force of advance decreases to create free rotation. The medical tube will not advance because of flexing in the position short of the hard portion. If the cup of the first end is penetrated, calcified content is likely to exist before the second end on a farther side. The tubular tip is captured and rotates freely to cause failure in the advance.

### SUMMARY OF THE INVENTION

In view of the foregoing problems, an object of the present invention is to provide a medical instrument and medical equipment for treatment, and a rotational handle device, in which rotation can be transmitted to a distal end or tubular tip by preventing application of excessive torque.

In order to achieve the above and other objects and advantages of this invention, a medical instrument for treatment in a tubular form is provided, including a tube for insertion by guiding of a medical guide wire inside. In the medical instrument, the tube includes a helical coil of metal. A tubular tip of metal is secured to an end of the helical coil. A cutting head of a blade shape is formed on the tubular tip, for cutting a lesion upon being rotated.

The tubular tip has a shape with a decreasing diameter toward a distal end thereof.

The tubular tip includes a U shaped cut or V shaped notch, formed in the cutting head at a front face and an outer face thereof, and directed in a direction along a tubular tip axis.

Alternatively, the cutting head includes a cut, formed to retreat from a front end face thereof, and having a cut surface inclined with reference to an axis of the tubular tip.

Also, the tubular tip includes a spiral groove formed in a tubular tip outer surface to extend helically from a front end face of the cutting head.

The helical coil is a multi filament helical coil constituted by helically wound plural wires of metal.

Alternatively, the helical coil is shaped by helically winding a wire after twisting plural strands of metal in a form of the wire.

Also, the helical coil is so wound as to decrease a diameter upon being rotated in a first rotational direction, and causes the cutting head to cut the lesion.

The helical coil has a shape with a decreasing diameter toward a distal end thereof.

The tubular tip is radiopaque.

Preferably, the medical instrument for treatment is endoluminal.

Furthermore, the medical instrument for treatment is endovascular.

Also, a rotational handle device rotates a medical instrument for treatment in a tubular form including a tube for insertion by guiding of a medical guide wire inside. The tube includes a helical coil of metal, a tubular tip of metal secured to an end of the helical coil, and a cutting head of a blade shape, formed on the tubular tip, for cutting a lesion upon being rotated. A first rotatable tube rotates with the tube, the first rotatable tube having a wire lumen for receiving insertion of the medical guide wire. An externally operable second rotatable tube is secured to the first rotatable tube in an axial direction. An over torque preventing mechanism is disposed between the first and second rotatable tubes, for transmitting rotation of the second rotatable tube to the first rotatable tube upon application of torque equal to or lower than a predetermined level to the second rotatable tube, and for causing the second rotatable tube to rotate free from the first rotatable tube upon application of torque higher than the predetermined level to the second rotatable tube.

One of the first and second rotatable tubes has a retaining projection, and a remaining one thereof has a retaining recess engageable with the retaining projection. The over torque preventing mechanism includes a tube support for keeping the first and second rotatable tubes movable in the axial direction between first and second positions, wherein the retaining projection retains the retaining recess when the first and second rotatable tubes are in the first position, and releases the retaining recess when the first and second rotatable tubes are in the second position. A biasing portion biases the first or second rotatable tube with a predetermined bias, retains the first and second rotatable tubes in the first position upon application of the torque equal to or lower than the predetermined level, to rotate the first and second rotatable tubes together, and sets the first and second rotatable tubes in the second position upon application of the torque higher than the predetermined level, to rotate the second rotatable tube free from the first rotatable tube.

The retaining projection is a plurality of first ridges formed on a tube end face of the first rotatable tube to extend radially. The second rotatable tube includes a plurality of second ridges formed on a tube end face thereof to extend radially, to define the retaining recess between, for mesh with the first ridges when the first and second rotatable tubes are in the first position, and for disengagement from the first ridges when the first and second rotatable tubes are in the second position.

The first and second ridges are teeth, and are in a triangular shape as viewed in a section perpendicular to a direction of extending radially.

The over torque limiting mechanism includes a first face gear formed on the first rotatable tube. A second face gear is formed on the second rotatable tube, for mesh with the first face gear. A biasing portion presses the second face gear on the first face gear. The second face gear is rotatable free from the first face gear when the torque applied to the first and second face gears becomes higher than the predetermined level.

Also, the first rotatable tube includes a shaft for supporting the second rotatable tube in a rotatable manner. The over torque preventing mechanism includes a shaft recess formed in the shaft. A lock ball is engageable with the shaft recess. A ball chamber contains the lock ball movably between a first position where the lock ball is shifted in the shaft recess and a second position where the lock ball is offset from the shaft recess. A biasing portion biases the lock ball with a predetermined bias, retains the lock ball in the first position upon application of the torque equal to or lower than the predetermined level, to rotate the first and second rotatable tubes together, and sets the lock ball in the second position upon application of the torque higher than the predetermined level, to rotate the second rotatable tube free from the first rotatable tube.

Furthermore, an adjuster adjusts the biasing portion in relation to the bias.

The second rotatable tube includes a first sleeve and a second sleeve having a smaller diameter than the first sleeve.

Also, a medical equipment for treatment includes a medical instrument for treatment in a tubular form including a tube for insertion by guiding of a medical guide wire inside. The tube includes a helical coil of metal, a tubular tip of metal secured to an end of the helical coil, and a cutting head of a blade shape, formed on the tubular tip, for cutting a lesion upon being rotated. A first rotatable tube rotates with the tube, the first rotatable tube having a wire lumen for receiving insertion of the medical guide wire. A connector removably connects the tube with the first rotatable tube. An externally operable second rotatable tube is secured to the first rotatable tube in an axial direction, for rotationally operating the medical instrument on a proximal side. An over torque preventing mechanism is disposed between the first and second rotatable tubes, for transmitting rotation of the second rotatable tube to the first rotatable tube upon application of torque equal to or lower than a predetermined level to the second rotatable tube, and for causing the second rotatable tube to rotate free from the first rotatable tube upon application of torque higher than the predetermined level to the second rotatable tube.

Also, a guide wire fitted medical equipment for treatment is provided as a composite equipment having the above devices.

In addition, a rotational handle device for rotationally operating a medical instrument for treatment on a proximal side is provided. The medical instrument in a tubular form includes a tube for insertion by guiding of a medical guide wire inside, wherein the tube includes a helical coil of metal, and a tip secured to an end of the helical coil. The rotational handle device includes a first rotatable tube for rotating with the tube, the first rotatable tube having a wire lumen for receiving insertion of the medical guide wire. An externally operable second rotatable tube is secured to the first rotatable tube in an axial direction. An over torque preventing mechanism is disposed between the first and second rotatable tubes, for transmitting rotation of the second rotatable tube to the first rotatable tube upon application of torque equal to or lower than a predetermined level to the second rotatable tube, and for causing the second rotatable tube to rotate free from the first rotatable tube upon application of torque higher than the predetermined level to the second rotatable tube.

### BRIEF DESCRIPTION OF THE DRAWINGS

The above objects and advantages of the present invention will become more apparent from the following detailed description when read in connection with the accompanying drawings, in which:

FIG. 1 is a plan illustrating a rotational medical instrument of the invention;

FIG. 2 is a horizontal section, partially broken illustrating a distal end part of the rotational medical instrument;

FIG. 3A is a perspective view illustrating Example 1 for a cutting edge of a tubular tip of the rotational medical instrument;

FIGS. 3B, 3C and 3D are perspective views illustrating plural structures of Example 1;

FIG. 4 is an explanatory view illustrating penetration of an occlusion with the rotational medical instrument;

FIG. 5 is an explanatory view illustrating treatment of an occlusion by the rotational medical instrument;

FIG. 6 is an explanatory view illustrating treatment of a lesion in a tortuous vessel by the rotational medical instrument;

FIG. 7A is a perspective view illustrating operation of the cutting edge of the tubular tip of the rotational medical instrument;

FIGS. 7B, 7C and 7D are views illustrating operation of plural examples of the cutting edge;

FIG. 8A is a perspective view illustrating Example 2 of the cutting edge of the tubular tip;

FIGS. 8B, 8C and 8D are perspective views illustrating plural structures of Example 2;

FIG. 9A is a side elevation illustrating Example 3 of the cutting edge of the tubular tip;

FIG. 9B is a front elevation illustrating the same as FIG. 9A;

FIGS. 9C and 9D are elevations illustrating one structure of Example 3;

FIGS. 9E, 9F, 9G and 9H are elevations illustrating plural structures of Example 3;

FIG. 10A is an explanatory view illustrating operation of the cutting edge of Example 3;

FIG. 10B is an explanatory view illustrating operation of another structure of Example 3;

FIG. 11A is a side elevation illustrating Example 4 of the cutting edge of the tubular tip;

FIG. 11B is a perspective view illustrating the same as FIG. 11A;

FIGS. 11C and 11D are an elevation and perspective illustrating one structure of Example 4;

FIG. 12A is a side elevation illustrating Example 5 of the cutting edge of the tubular tip;

FIG. 12B is a perspective view illustrating the same as FIG. 12A;

FIGS. 12C and 12D are an elevation and perspective illustrating one structure of Example 5;

FIG. 13 is a perspective view illustrating relationship between winding directions of the rotational medical instrument and a helical coil;

FIG. 14A is a horizontal section illustrating Example 6 of the helical coil of the rotational medical instrument;

FIG. 14B is a horizontal section, partially broken illustrating another structure of Example 6;

FIG. 15A is a horizontal section illustrating a pitch of winding in the helical coil;

FIG. 15B is a horizontal section illustrating a pitch of winding in a multi filament helical coil in Example 6 in comparison with FIG. 15A;

FIG. 16 is a horizontal section illustrating a multi filament helical coil containing plural wires of two or more wire diameters;

FIG. 17A is a horizontal section illustrating Example 7 for the helical coil of the rotational medical instrument;

FIG. 17B is a horizontal section illustrating one structure of Example 7;

FIG. 18 is a perspective view illustrating Example 8 for the tubular tip of the rotational medical instrument;

FIG. 19A is an explanatory view illustrating withdrawal of a balloon catheter by use of the rotational medical instrument;

FIG. 19B is an explanatory view illustrating a step included in the withdrawal and next to a step of FIG. 19A;

FIG. 20 is an explanatory view illustrating a step of perforating a balloon catheter;

FIG. 21A is a front elevation illustrating operation of the embodiment in which the winding directions are different between the helical coil of the rotational medical instrument and that of the guide wire;

FIG. 21B is an explanatory view illustrating the helical coil of the rotational medical instrument;

FIG. 21C is an explanatory view illustrating the helical coil of the guide wire;

FIG. 22 is a perspective view illustrating an over torque preventing mechanism of the rotational medical instrument;

FIG. 23 is a horizontal section illustrating the same as FIG. 22;

FIG. 24 is a perspective view, partially cutaway illustrating one preferred over torque preventing mechanism;

FIG. 25A is an explanatory view illustrating an example of gear teeth;

FIGS. 25B and 25C are explanatory views illustrating plural examples of gear teeth with changes in angles;

FIG. 26 is an explanatory view illustrating treatment of an occlusion by use of a rotational medical instrument known in the art.

### DETAILED DESCRIPTION OF THE PREFERRED EMBODIMENT(S) OF THE PRESENT INVENTION

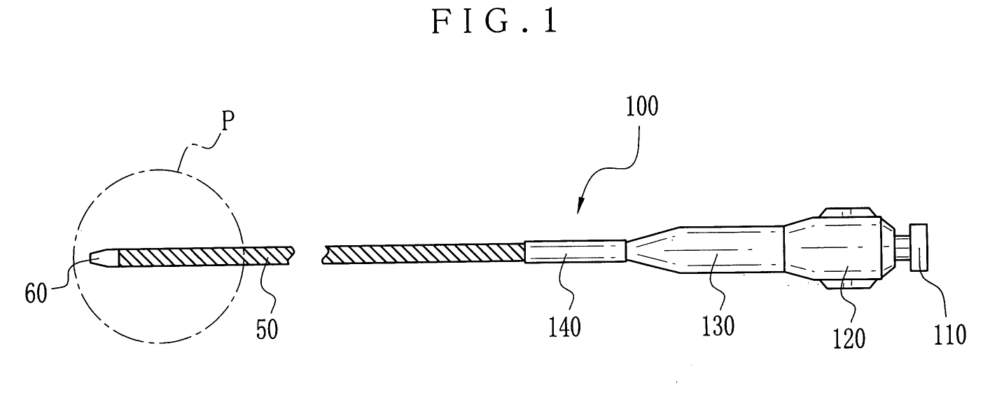

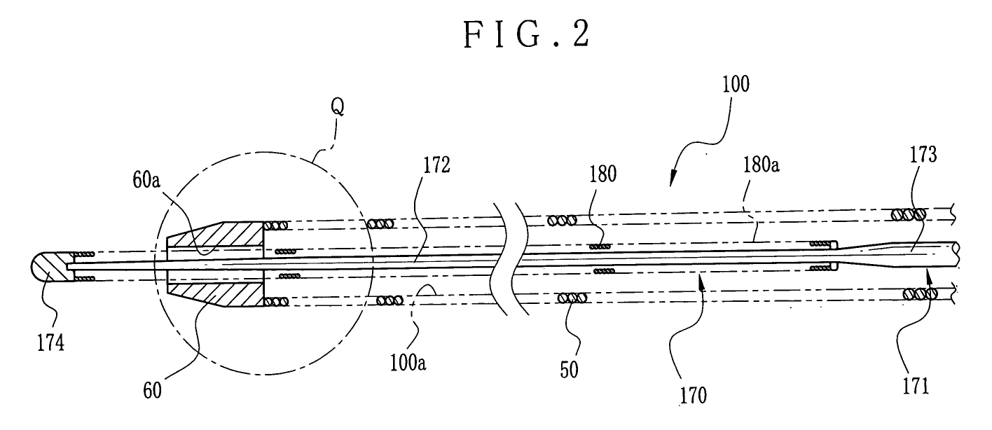

A preferred embodiment of the invention is described by referring to the drawings. In FIG. 1, appearance of a rotational medical instrument 100 or cutting instrument in a catheter shape is illustrated. The rotational medical instrument 100 of FIG. 1 has a hollow tubular shape, in which a lumen or through hole is formed from end to end. The rotational medical instrument 100 includes a connector 120, a protector 130, a protective tube 140, a tubular winding or helical coil 50 and a tubular tip 60 in an order toward a distal end. The connector 120 has a Luer or screw 110. When the rotational medical instrument 100 is used for medical use, the method is similar to the use of a vascular catheter. A medical guide wire 170 of FIG. 2 is inserted or introduced at first in a blood vessel. The rotational medical instrument 100 is then inserted by following the guide wire 170. The rotational medical instrument 100 is thrust into a constriction of a lesion, and then is rotated or advanced, so a space at constriction is enlarged, or atheroma or tissue in an occlusion is cut and removed.

In FIG. 2, a distal end part of the rotational medical instrument 100 of the invention is illustrated in a region with a predetermined length. This is the region P in FIG. 1. The guide wire 170 stands inserted in the rotational medical instrument 100 in the drawing. A lumen or through hole 100a extends through the rotational medical instrument 100, so the guide wire 170 in FIG. 2 guides the rotational medical instrument 100 while inserted in the lumen 100a. The guide wire 170 includes a wire 171 and a tubular winding or helical coil 180. A tapered wire shaft 172 of the wire 171 has a reduced diameter. The helical coil 180 is disposed about the tapered wire shaft 172 to cover the same. A proximal wire shaft 173 is a base included in the wire 171. The wire 171 has such a shape that the proximal wire shaft 173 has a diameter of 0.355 mm. The wire 171 is one part of stainless steel or nickel-titanium alloy, and has a length of 1.5-3 meters. The tapered wire shaft 172 has a total length of 110-150 mm, and a reduced diameter of 0.142 mm. A distal end of the tapered wire shaft 172 is has a further reduced diameter with a length of 40-50 mm.

An end part cap 174 is fixed on a distal end of the tapered wire shaft 172 of the guide wire 170 by attachment with wax or the like. A proximal coil end 180a of the helical coil 180 of the guide wire 170 is fixed on the wire 171 by attachment with wax or the like. A distal end of the helical coil 180 is connected with the end part cap 174 by attachment with wax or the like. The wire 171 having the end part cap 174 is previously inserted in a blood vessel of a human body by manually operating a base of the proximal wire shaft 173 by way of a guide for diagnosis of a state of a vascular lesion or for treatment, such as atherectomy. The helical coil 180 is wound about the wire 171, and is a roll of a wire of metal having a wire diameter of 0.065 mm at a roll diameter of 0.355 mm.

In the rotational medical instrument 100 of FIG. 2, a region Q has a predetermined length from a distal end thereof, and includes the helical coil 50 and the tubular tip 60 fitted thereon. In FIG. 3A, a region Q is depicted in enlargement. A cutting head or blade 10 is included in the tubular tip 60. Also, the lumen 100a is formed to extend through the rotational medical instrument 100 including the distal end part of FIG. 2, to define a catheter shape or tubular shape for receiving insertion of the guide wire 170. The tubular tip 60 includes an opening 60a, and has a cylindrical shape in a straight manner or a diameter decreasing shape in a tapered manner, or has a shape defined by combination of such partial shapes. Preferred examples of materials of the tubular tip 60 are gold, platinum, wax, or other radiopaque substance which is impermeable to radioactive radiation.

In the embodiment, the cutting head 10 is positioned at the end of the tubular tip 60. This is a composite shape including a sharp end portion for initially advancing into an occlusion as a lesion in a blood vessel, and a cutting blade portion for cutting the occlusion upon rotation. In FIGS. 1 and 2, the cutting head 10 is used as an element of a rotational medical instrument for treatment in a tubular form. The following is effects of the feature of the invention.

First, there is an effect even when the guide wire 170 cannot come through because of firmly calcified thrombus in the total occlusion of a coronary artery lesion. It is possible in the rotational medical instrument 100 to start cutting or shearing the occlusion with the cutting head 10, and to perforate the occlusion, so that a true lumen of the artery can be reached to smooth an inner wall readily by penetrating a blood vessel.

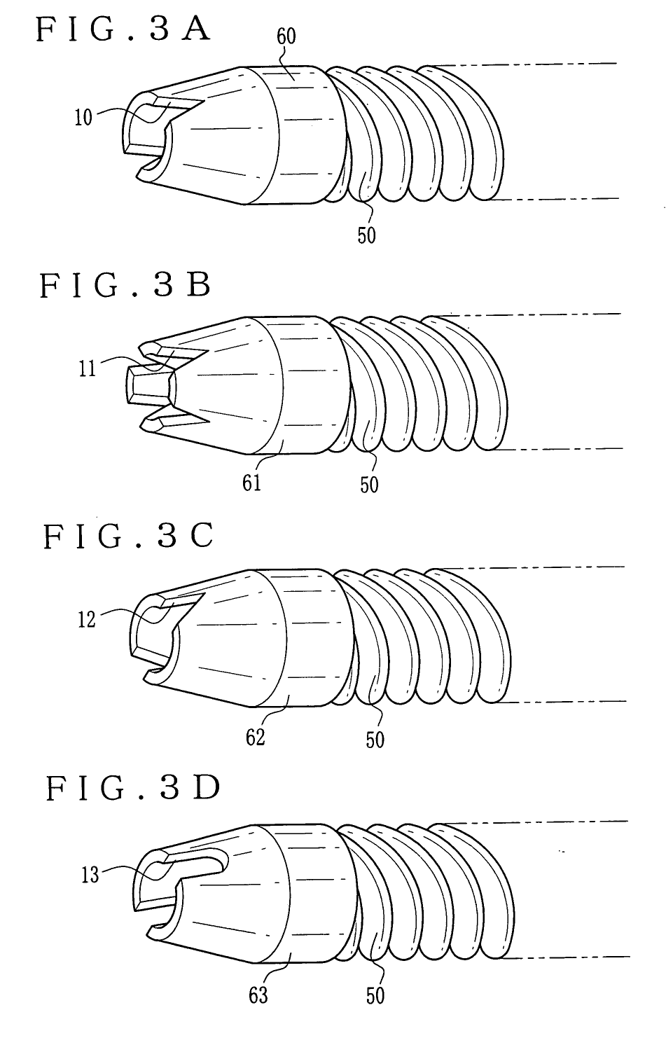

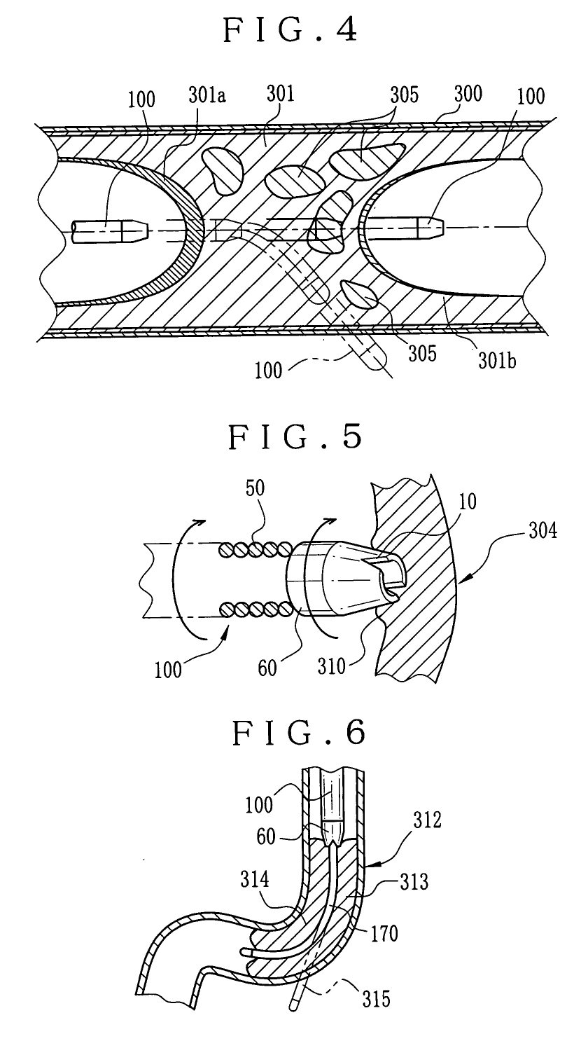

In FIG. 4, advance of the rotational medical instrument 100 into an occlusion is illustrated. A blood vessel 300 has a total occlusion 301. In FIG. 26, failure in advance of a rotational medical instrument 400 or cutting instrument in a catheter shape into an occlusive lesion 401 is illustrated. In FIG. 5, the rotational medical instrument 100 advances into an occlusive lesion 304. In FIG. 4, the total occlusion 301 in widely observed cases is illustrated, and has two ends in a cup shape. A first end 301a on a nearer side has two end points with a great thickness, so that a true lumen can be reached easily. A difficulty lies in that the total occlusion 301 cannot be penetrated readily due to a considerable thickness of the hard portion. A second end 301b on a farther side has a portion with high hardness and small thickness. However, a middle point of the second end 301b protrudes convexly, so that a cutting edge is likely to offset from the middle. This problem is serious in particular if calcified content 305 with high hardness is contained in advance of the second end 301b. A direction of advance of the rotational medical instrument 100 is likely to alter by following a softer portion in atheroma or tissues. False lumens may be reached and also penetration of a blood vessel may occur as indicated by the phantom line.

In FIG. 26, a known type of the rotational medical instrument 400 is illustrated. The rotational medical instrument 400 has a tubular winding or helical coil 403, and a tubular tip 402 secured to an end of the helical coil 403. The rotational medical instrument 400 does not have the cutting head 10 of the invention, and does not operate for cutting, because shaped cylindrically to have a regular form as viewed in a cross section. A first end of the occlusive lesion 401 has a hard portion 401a. The advance of the tubular tip 402 is weaker according to greatness of a thickness of the hard portion 401a, so the tubular tip 402 may rotate with slip in the hard portion 401a. The helical coil 403, as indicated by the broken line, is deformed elastically and cannot advance further. Even when the tubular tip 402 can thrust the first end of the occlusive lesion 401, calcified content is likely to exist in a position short of the second end, and to hinder the tubular tip 402 from advancing as the tubular tip 402 is captured and caused to rotate with slip.

However, it is possible with the rotational medical instrument 100 to prevent the above-described failure of slipped rotation or the like. FIGS. 4 and 5 are referred to now. A distal end part and proximal end part of the tubular tip 60 in FIG. 5 are different from one another in the shape of the section. The tubular tip 60 is tapered to decrease the area of the section, so a surface pressure increases. When the tubular tip 60 is pressed on a hard portion 310, the cutting head 10 operates as a sharp end portion to advance into the hard portion 310. A hard component of the occlusion is sheared, and split to extend on the cutting head 10. The rotational medical instrument 100, in operation as a cutting blade portion, is rotated to cut away the hard component of the atheroma or tissues. The operation is repeated similarly. Thus, the perforation and advance in a continuous manner are possible irrespective of varied forms of lesions.

It is possible in the rotational medical instrument 100 of FIG. 4 to smooth an inner wall of a lumen upon thrusting the total occlusion 301 and after penetrating the same. Passage of a balloon catheter and stent-fitted balloon catheter can be facilitated in a lesion. It is easy to introduce a catheter to a target position, and to extend a space in a lesion for a suitable position, and to deploy and leave a stent. An effect in acquiring a smooth surface is because of rotational cutting of a cutting blade similar to that of a drill as a tool for perforation, and because of the shape of the tubular tip 60 smooth and equiradial. Furthermore, the tubular tip 60 can be preferably provided with a polished or abraded surface to form a smooth surface.

In FIG. 6, a total occlusion 312 in the coronary artery receives thrusting of the cutting head 10 of the rotational medical instrument 100. When the rotational medical instrument 100 is kept thrust in the total occlusion 312, support can be reinforced by backup for advance of the guide wire 170. See FIG. 2. Even when the guide wire 170 in use is the same, the backup support can raise the force of penetration in an occlusion 313. In the known instrument with the regularly cylindrical hollow tip, slipped rotation of the instrument is likely to occur in a circumferential highly calcified lesion of which the total occlusion 312 is hardened nearly totally, a superficial calcified lesion, and a diffuse lesion having a length of 25 mm or more.

The reason for this is that the cutting head 10 of the invention does not exist in the prior art. Even when force is applied to advance the guide wire in the known structure, this cannot be supported as reaction of the force of the push in the above state with a slip. The force operates in reverse, and may move back the tip to offset the tip from the occlusion.

In FIG. 6, a state of treating the total occlusion 312 in a tortuous form by use of the rotational medical instrument 100, in comparison with a state with a known guide wire 315 indicated by the phantom line. The guide wire 170 for penetration of the total occlusion 312 of FIG. 2 preferably has a high rigidity in flexure at its end portion, for example with a diameter as much as 0.1 mm. In FIG. 6, an intractable case is illustrated, in which a tortuous portion 314 exists on a farther side of the total occlusion 312. The known guide wire 315 with the high rigidity cannot flex sufficiently, to cause perforation of a blood vessel as indicated by the phantom line. This is typical in a lesion in the bifurcation of the right coronary artery.

In the rotational medical instrument 100 of the embodiment, the tubular tip 60 is caused to thrust in a first end of the total occlusion 312 which is extremely flexed or curved. Force of reaction to advance the guide wire 170 (in FIG. 2) can be supported by the retention of the tubular tip 60 and the first end by the thrusting. If the guide wire has a tip of a distal end having a small rigidity of flexing, its force of the penetration can be a plurality of times as high as before. Introduction through the tortuous portion 314 is possible owing to the flexible guide wire in a smooth manner. As an effect, backup support can be ensured to reinforce pressure of the guide wire.

Various structures and forms can be used in the invention for the cutting head 10 of the tubular tip 60 of the rotational medical instrument 100, or the helical coil 50 or the tubular tip 60. Preferred examples will be hereafter described.

### EXAMPLE 1

In FIGS. 3A-3D, cutting heads or blades of tubular tips of Example 1 are illustrated, the structures including a first structure of FIG. 3A, a second structure of FIG. 3B, a third structure of FIG. 3C, and a fourth structure of FIG. 3D. The tubular tip 60 has the cutting head 10 with a surface to extend toward the axis. A tubular tip 61 has a cutting head or blade 11. Also, tubular tips 62 and 63 have cutting heads or blades 12 and 13. In FIG. 3A, the first structure has the cutting head 10 constituted by two V shaped notches positioned opposite to one another relative to the center. In FIG. 3B, the second structure has the cutting head 11 constituted by four V shaped notches positioned regularly at four points defined by equally dividing the circumference. In FIG. 3C, the third structure has the cutting head 12 constituted by inclined V shaped notches formed at two points at the end of the tubular tip 62. In FIG. 3D, the fourth structure has the cutting head 13 constituted of a U shaped cut in place of the cutting head 10 of the first structure. Note that a U shaped cut may be formed in FIGS. 3B and 3C in place of the cutting heads 11 and 12.

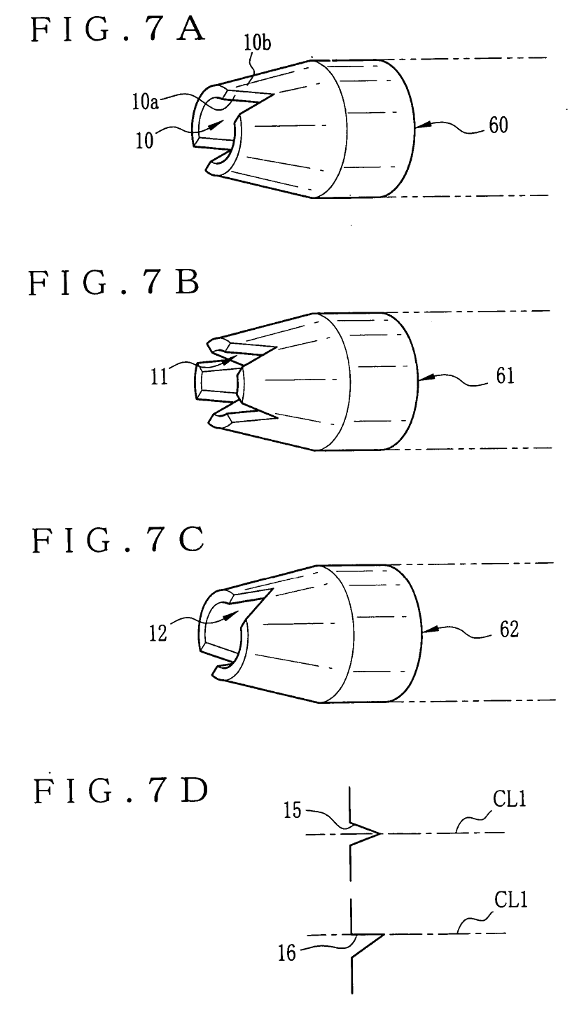

In FIGS. 7A-7D, effect of Example 1 is illustrated. A V shaped notch or U shaped cut in the tubular tips 60-62 constitutes the cutting heads 10-12. In FIG. 7A, the cutting head 10 has a symmetrical form. A cutting edge 10a for thrusting has the V shaped notch. A cutting edge lob for shearing is constituted by a side wall in a V shape. The tubular tips 60-62 have a shape with a decreasing diameter toward the distal end in view of its area of a section. An end face has a sharp end portion. A cutting blade portion is formed in the longitudinal direction for cutting in a rotational direction. Force of thrusting and cutting a lesion can be raised. An atheroma or tissues can be penetrated and perforated easily even if with high hardness. Also, a V shaped notch or U shaped cut are formed to raise thrusting force.

To be precise, an area of the cross section of the distal end is reduced to set high a pressure of the surface, to increase force for entry into a lesion. As cutting blades are defined by V shaped or U shaped walls, protruding tissues can be removed by rotating the cutting blades. If the number of the V shaped grooves is set greater, suitability for trusting into the lesion and for removal of the same can be higher. As distal ends of the tubular tips 60-63 are formed with a decreasing diameter, transmission of torque to the distal ends can be efficient. A ratio of torque depends upon a ratio between radii of rotating points relative to an axis. In Example 1, the tubular tips 60-63 have a larger diameter of 1 mm on the proximal end side, and a smaller diameter of 0.75 mm on the distal end side. The torque can be increased by approximately 1.33 times as much.

In FIG. 7D, two types of V shaped notches 15 and 16 are illustrated. The V shaped notch 15 is an A type of an isosceles triangle with reference to the axis CL1 of the tubular tip. The V shaped notch 16 is a B type of which one of two surfaces is parallel to the axis CL1 of the tubular tip. In FIGS. 7A and 7B, the cutting heads 10 and 11 are constituted by the A type or the V shaped notch 15. In FIG. 7C, the cutting heads 10 and 11 are constituted by the B type or the V shaped notch 16. Also, a U shaped cut can be a selected one of two types.

In the type B, a first one of two walls of the V shaped notch or U shaped cut is disposed to extend in parallel with the axial direction or with the generating line. This provides a difference in the cutting ability in such a manner that cutting ability is high in rotation in a first direction and that a cutting ability is lower in rotation in a second direction. It is possible for a physician to select one of the directions according to a phase of a lesion between high and low cutting abilities. In Example 1, passage or introduction of a balloon catheter can be facilitated in a lesion because of forming a smooth surface by penetration. Backup support can be ensured to reinforce pressure of the guide wire.

### EXAMPLE 2

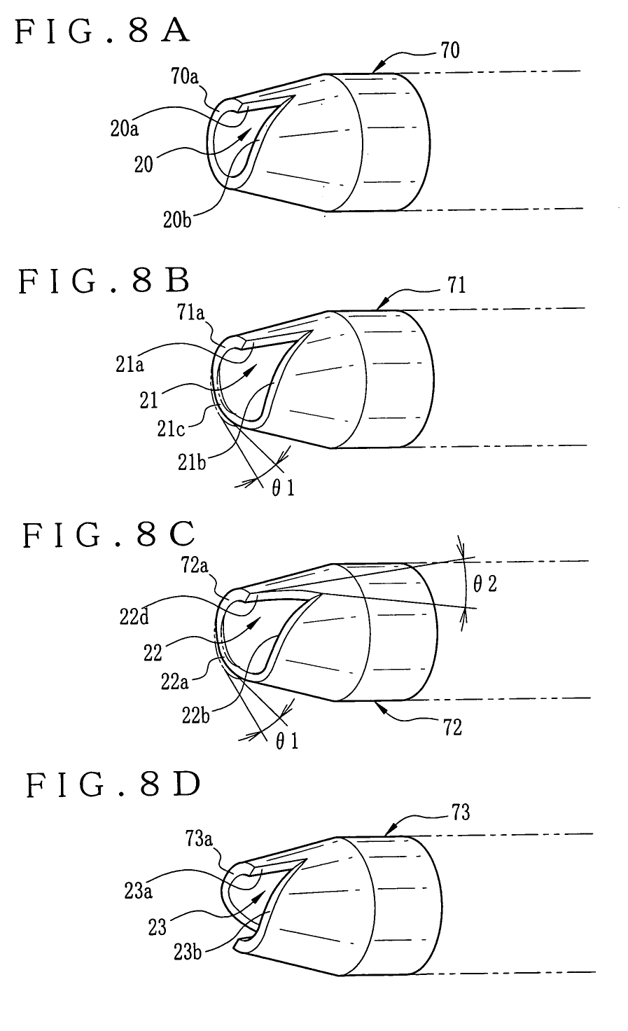

In FIGS. 8A-8D, cutting heads or blades of tubular tips of Example 2 are illustrated in a perspective view, the structures including a first structure of FIG. 8A, a second structure of FIG. 8B, a third structure of FIG. 8C, and a fourth structure of FIG. 8D. Cutting heads or blades 20, 21, 22 and 23 are inclined cutting edges relative to an axial direction. In FIG. 8A, a first structure is illustrated. The cutting head 20 has a cutting edge 20a, which extends along a straight line contained in one plane passing the axis and at one point on an end face 70a of a tubular tip 70. A cutting edge 20b is inclined relative to the axial direction and opposed to the cutting edge 20a.

In FIG. 8B, a second structure is illustrated. The cutting head or blade 21 has an end inclined cutting edge 21c, which is defined by recessing the cutting edge 20b of the cutting head 20 in FIG. 8A. A tubular tip 71 has an end face 71a. A cutting edge 21a lies at a point on the end face 71a and extends in a direction along a generating line which is coplanar with the axis. A cutting edge 21b is inclined and opposed to the cutting edge 21a. The end inclined cutting edge 21c is formed at the end of the cutting edge 21b. In FIG. 8C, a third structure is illustrated. The cutting head or blade 22 has an inclined cutting edge 22d, which is defined by inclining the cutting edge 21a of the cutting head 21 of FIG. 8B at an angle θ2 with reference to the generating line. A tubular tip 72 has an end face 72a. An inclined cutting edge 22a lies at a point on the end face 72a, and extend with an inclination at an angle θ1 from the radial direction. A cutting head or blade 22b extends from the inclined cutting edge 22a. The inclined cutting edge 22d is opposed to the inclined cutting edge 22a and the cutting head 22b.

In FIG. 8D, a fourth structure is illustrated. The cutting head or blade 23 has a cutting edge 23a which extends along a generating line, namely straight line contained in one plane passing the axis and at each one of two points on an end face 73a of a tubular tip 73. A cutting edge 23b is inclined relative to the axial direction and opposed to the cutting edge 23a. The tubular tip 73 is different from the tubular tip 62 in FIG. 3C in that the cutting edges 23a and 23b intersect with one another to define an angular end shape. Also, the cutting edge 23b is helically shaped. The fourth example has a drill shaped blade.

The tubular tips 70-72 have the cutting heads 20-22 extending with an inclination from the distal end in a direction toward the longer axis. The tubular tip 73 has the cutting head 23 in a drill shape. Any one of the cutting heads 20-23 includes a sharp end portion and a cutting blade portion. Those structures are evaluated for performance and ranked according to ability of thrusting and cutting to a lesion. The fourth structure of FIG. 8D has the highest ability. The second and third structures of FIGS. 8B and 8C have a second highest ability lower than that of the fourth structure. The first structure of FIG. 8A has the lowest ability lower than that of the second and third structures. An area of the tubular tip 73 of the cutting head 23 viewed in a section is the smallest among the tubular tips 70-73. Also, cutting ability of the tubular tip 73 of the cutting head 23 by rotation is the highest. This is because two main cutting edges extend perpendicular to the rotational direction unlike the other structures having one main cutting edge.

Thus, the cutting ability can be raised by forming the cutting blade portion parallel with the axis. Also, the end faces are inclined at the angles θ1 and θ2 toward the proximal end in the second and third structures, to raise ability of penetration and advance. In Example 2, passage or introduction of a balloon catheter can be facilitated in a lesion because of forming a smooth surface by penetration. Backup support can be ensured to reinforce pressure of the guide wire, in a manner similar to the above example.

### EXAMPLE 3

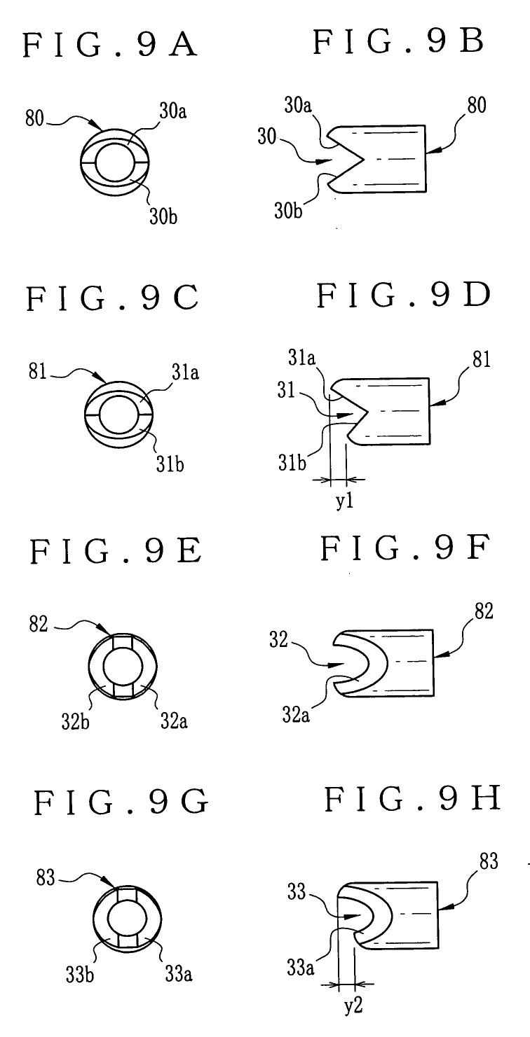

In FIGS. 9A-9H, cutting heads or blades of tubular tips of Example 3 are illustrated in a side elevation and front elevation, the structures including a first structure of FIGS. 9A and 9B, a second structure of FIGS. 9C and 9D, a third structure of FIGS. 9E and 9F, and a fourth structure of FIGS. 9G and 9H. In FIGS. 9A and 9B, a tubular tip 80 has a cutting head or blade 30. Notch formed cutting edges 30a and 30b are disposed on the cutting head 30, and are symmetrical with one another with reference to a centerline passing the center of the tubular tip 80. In FIGS. 9C and 9D, a tubular tip 81 has a cutting head or blade 31. Notch formed cutting edges 31a and 31b are disposed on the cutting head 31, and are asymmetrical with one another with reference to a centerline passing the center of the tubular tip 81. In FIGS. 9E and 9F, a tubular tip 82 has a cutting head or blade 32. Crescent shaped cutting edges 32a and 32b are disposed on the cutting head 32, and are symmetrical with one another with reference to a centerline passing the center of the tubular tip 82. In FIGS. 9G and 9H, a tubular tip 83 has a cutting head or blade 33. Crescent shaped cutting edges 33a and 33b are disposed on the cutting head 33, and are asymmetrical with one another with reference to a centerline passing the center of the tubular tip 83.

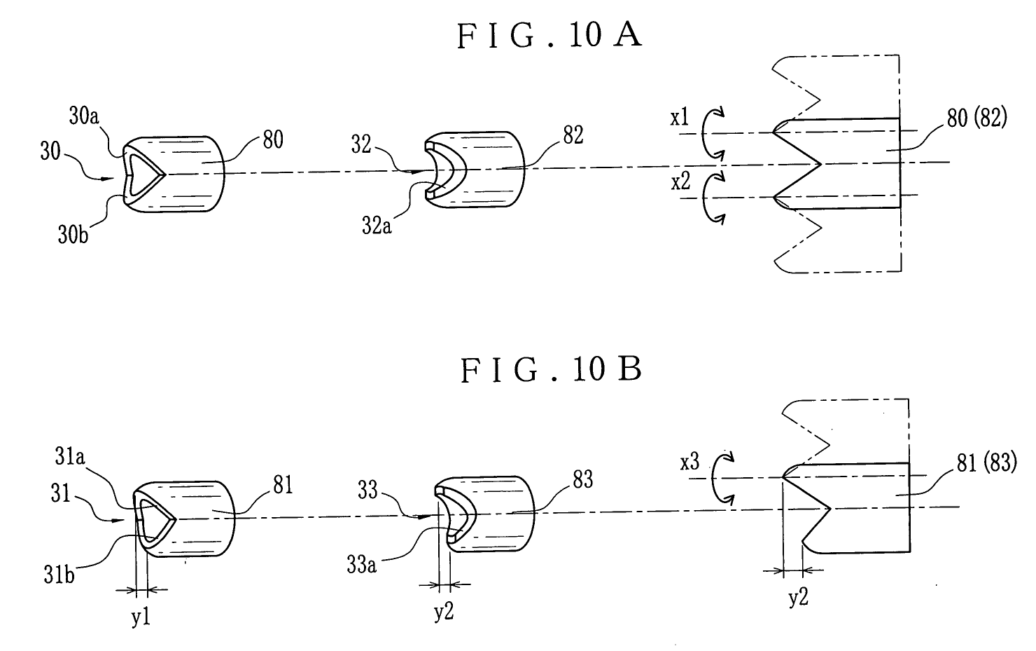

In FIGS. 9A and 9B, effects of the tubular tips 80-83 are illustrated. The tubular tips 80 and 81 have the cutting heads 30 and 31. The tubular tips 82 and 83 have the cutting heads 32 and 33. The blade structures can keep hard tissue picked up. The cutting blade portion can cut the lesion without releasing the hard tissue. In the first and third structures of FIGS. 9A and 9B and FIGS. 9E and 9F, the product rotates about a selected one of axes x1 and x2 in FIG. 10A. This is effective in enlarging the perforating diameter unlike the above embodiment.

In contrast, the second structure of FIGS. 9C and 9D and the fourth structure of FIGS. 9G and 9H are rotational about a single axis of x3 as indicted in FIG. 10B because of the differences y1 and y2 in the position between two projecting portions. Rotational positions can be stabilized higher than the first and third structures in addition to enlargement of the perforating diameter of the distal end. Therefore, a suitable structure can be selected among those by considering phases of lesions, so a lesion can be treated in a medically optimized method. In Example 3, passage or introduction of a balloon catheter can be facilitated in a lesion because of forming a smooth surface by penetration. Backup support can be ensured to reinforce pressure of the guide wire, in a manner similar to the above examples.

### EXAMPLE 4

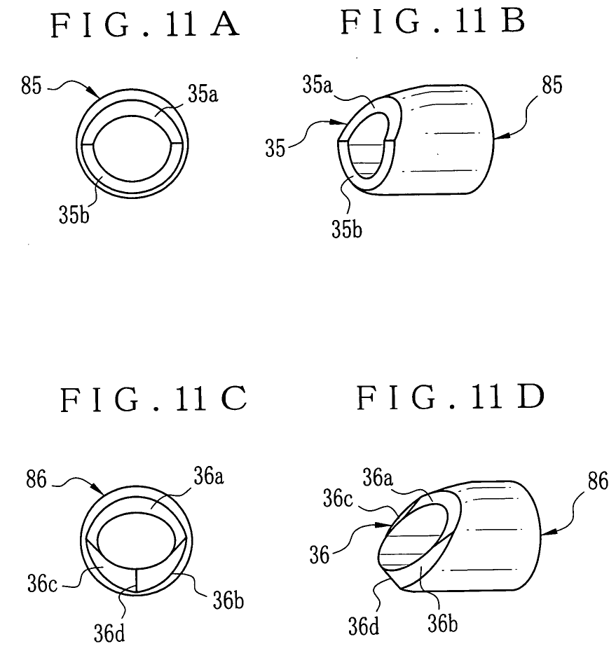

In FIGS. 11A-11D, cutting heads or blades of tubular tips of Example 4 are illustrated in a side elevation and perspective view, the structures including a first structure of FIGS. 11A and 11B, and a second structure of FIGS. 11C and 11D. In FIGS. 11A and 11B, a tubular tip 85 has a decreasing diameter. An arc shaped inclined cutting head or blade 35 of the tubular tip 85 has an inclined cutting edge 35a and a vertical end face 35b. In FIGS. 11C and 11D, a tubular tip 86 has an inclined face 36a, and knife edge faces 36b and 36c which are obtained by cutting with an inclination from the front face. Also, a knife cutting edge 36d is formed on the tubular tip 86 at a point crossing the knife edge faces 36b and 36c.

In FIGS. 11A and 11B, an end face of the first structure is defined by the inclined cutting edge 35a and the vertical end face 35b. An area of the cross section of this gradually decreases in a direction toward the front end, to determine surfaces for thrusting and slitting. In FIGS. 11C and 11D, the faces 36a-36c of the second structure are inclined. The knife cutting edge 36d is defined by the knife edge faces 36b and 36c, so that force for penetration and thrusting can be higher in tearing issue in a lesion. Note that the knife cutting edge 36d is so shaped that its inner peripheral side protrudes to the front in comparison to the outer peripheral side. This is because walls of a blood vessel would be likely to be penetrated if the knife cutting edge 36d were inclined in an opposite direction, namely its outer peripheral side protruded to the front in comparison to the inner peripheral side.

Note that selection between the first and second structures of FIGS. 11A-11D is determined according to hardness of a lesion, a tortuous shape of a blood vessel, or other states of a lesion. The tubular tips 85 and 86 have a shape with a decreasing diameter toward the distal end in view of its area of a section. An end face has a sharp end portion. A cutting blade portion is formed in the longitudinal direction for cutting in a rotational direction. Force of thrusting and cutting a lesion can be raised, to penetrate and perforate an atheroma or tissues easily. In Example 4, passage or introduction of a balloon catheter can be facilitated in a lesion because of forming a smooth surface by penetration. Backup support can be ensured to reinforce pressure of the guide wire, in a manner similar to the above examples.

### EXAMPLE 5

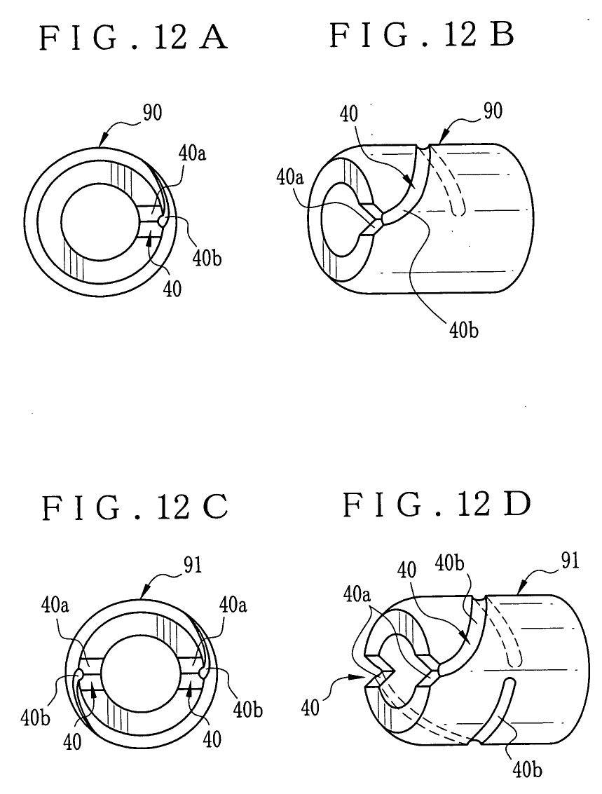

In FIGS. 12A-12D, cutting heads or blades of tubular tips of Example 5 are illustrated in a side elevation and perspective view, the structures including a first structure of FIGS. 12A and 12B, and a second structure of FIGS. 12C and 12D. In FIGS. 12A and 12B, a tubular tip 90 has a cutting head or blade 40. A V shaped notch 40a of the cutting head 40 is formed in a front face of the tubular tip 90. A spiral groove 40b is formed to extend from the V shaped notch 40a. In FIGS. 12C and 12D, a tubular tip 91 has a pair of the cutting heads 40 positioned rotationally with a difference of a flat angle.

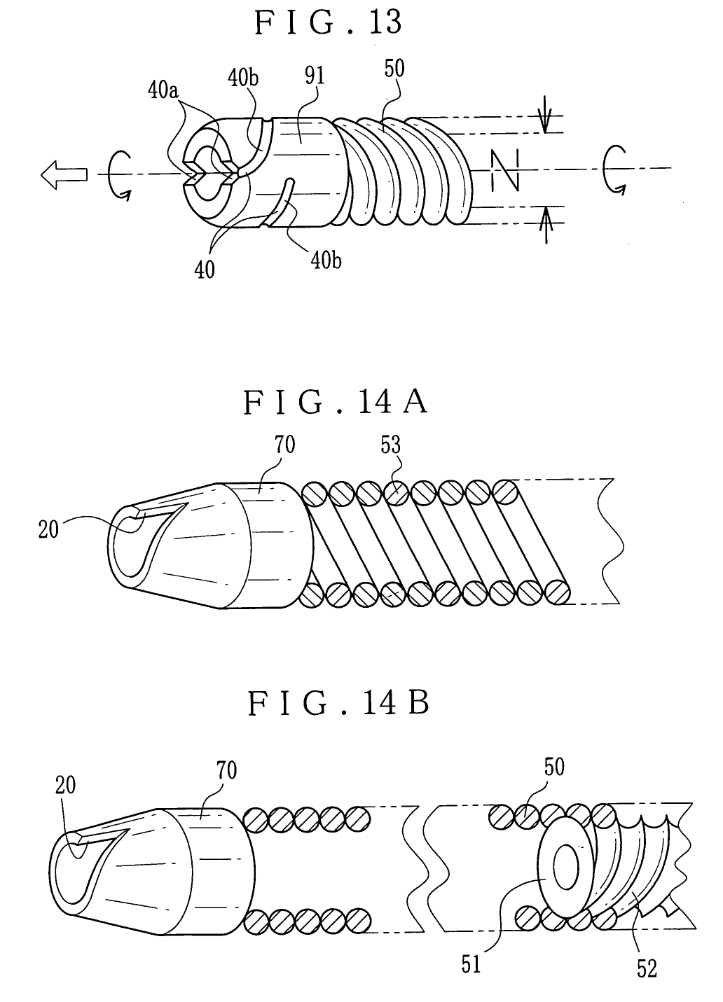

In FIG. 13, effects of Example 5 with the cutting head 40 of the tubular tip 91 are illustrated. A direction of winding of the helical coil 50 is correlated with a rotational direction of cutting. The V shaped notch 40a is formed in a face of the tubular tip 91. The spiral groove 40b extends from the V shaped notch 40a and formed outside. This raises ability of thrusting into a lesion. The spiral groove 40b makes it easy to advance into a hard lesion owing to cutting ability upon rotation by the cutting blade portion. Also, a plurality of the spiral grooves 40b can raise this ability.

In FIG. 13, turns of the spiral groove 40b are in the left screw direction. Turns of the helical coil 50 are in the right screw direction or Z direction, which is effective in high performance in penetration. When rotational force is applied in the counterclockwise direction, the helical coil 50 becomes more rigid owing to the direction of reducing the diameter by twisting. Also, the tubular tip 91 is thrust when rotated in the left screw direction, to advance and cut with high force. This is particularly effective when the lesion is hard. In Example 5, passage or introduction of a balloon catheter can be facilitated in a lesion because of forming a smooth surface by penetration. Backup support can be ensured to reinforce pressure of the guide wire, in a manner similar to the above examples.

### EXAMPLE 6

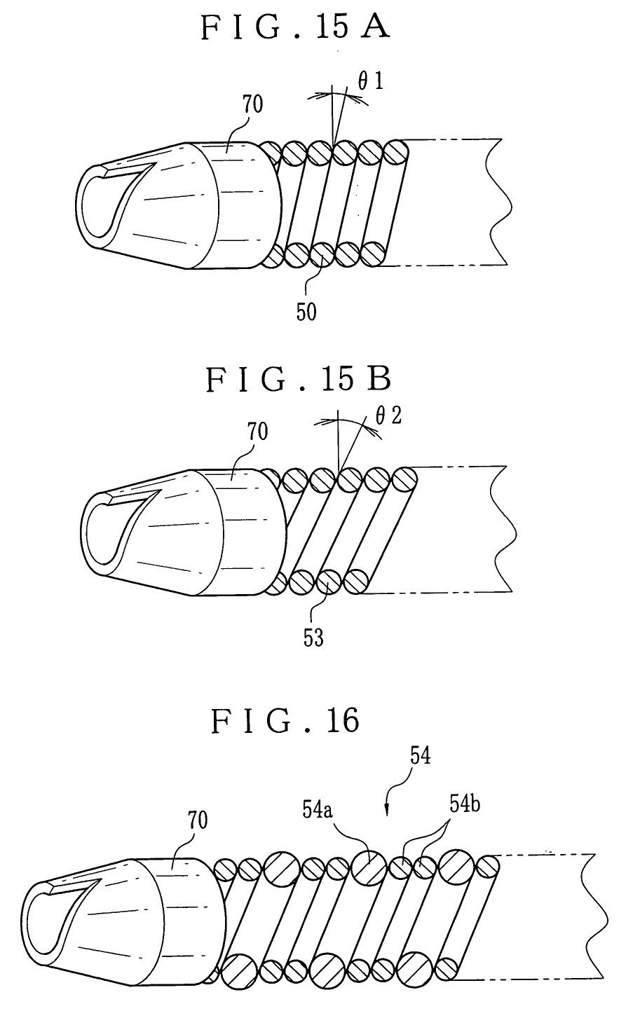

In FIGS. 14A and 14B, tubular windings or helical coils of Example 6 are illustrated, the structures including a first structure of FIG. 14A, and a second structure of FIG. 14B. In FIG. 14A, a multi filament tubular winding or helical coil 53 is used in combination with the tubular tip 70 of each one of above described embodiments or examples. In FIG. 14B, a metal tube 51 has a spiral groove 52. A proximal end of the helical coil 50 is secured to the spiral groove 52. In FIGS. 15A and 15B, the pitch of turns of the helical coil 50 of Example 6 is illustrated in comparison with a single wire helical coil. In FIG. 16, the multi filament helical coil of Example 6 with wires of different diameters is illustrated.

It is possible according to the multi filament structure of the multi filament helical coil 53 to raise transmission of torque to the tubular tip 70 and to increase ability of cutting and penetrating of the tubular tip 70. In contrast with the helical coil 50 in which a single wire transmits rotation from end to end, the multi filament helical coil 53 has plural wires transmitting rotation. A pitch of turns is greater according to the plural number of the wires. An angle θ2 of inclination is greater to facilitate transmission of rotation.

A specific example of the multi filament helical coil 53 is a structure of 8-12 wires having a wire diameter of 0.16 mm and wound at a roll diameter of 1.0 mm. It is possible to wind a great number of wires about a mandrel to obtain a helical coil. To this end, a mandrel winding method is used. Furthermore, a plurality of strands can be twisted to obtain wires, which can be wound to obtain a helical coil. For this, a strand twisting method in a manner of rope making is used. The mandrel winding method is likely to create gaps between turns of the plural wires, due to shortage in initial tension of wires. The strand twisting method is further preferable owing to prevention of such gaps. Examples of materials of wires can be one or more of stainless steel, tungsten, nickel-titanium alloy, gold, platinum and other radiopaque material.

In FIGS. 15A and 15B, the angle θ2 of the inclination of the wires is kept high by keeping high the pitch of turns by use of the multi filament helical coil 53 of a multi filament form including plural wires. Also, a distance of moving the tubular tip 60 per one rotation can be increased. As the pitch of turns becomes greater, it is possible to keep efficiency high in movement and removal because of greater distance of removed debris of atheroma or tissues toward the rear. In FIG. 16, another preferred tubular winding or helical coil 54 is illustrated, and is constituted by a large diameter wire 54a and a small diameter wire 54b in combination. The removed debris of atheroma or tissues are moved and removed in gaps between wires in the helical coil 54. The effect of this is higher according to highness of the pitch of turns.

In FIG. 14B, the metal tube 51 is disposed on the proximal end of the helical coil 50, and is effective in making efficient transmission of torque to the tubular tip 70. The number of rotations of the helical coil 50 must be high at the proximal end according to greatness of its length in view of reliable transmission of rotations to the tubular tip 70. This is because the angle of turns is proportional to the number of turns of the coils, and is greater according to the greatness of the length of the helical coil 50.

The metal tube 51 is connected on a proximal end side of the helical coil 50. The number of turns of the helical coil 50 is reduced by shortening the helical coil 50. This causes reduction in the angle of turns. The number of rotations can be lowered, so transmission of rotations to the tubular tip 70 can be ensured. Also, rigidity on the proximal end side is higher, to improve a characteristic of thrusting. For example, the helical coil 50 is 1,200 mm long. The metal tube 51 is 300 mm long on the proximal end side. The number of rotations for transmitting rotation to the tubular tip 70 can be lower than that according to the former examples by a proportion of approximately ¼. It is advantageous to reduce fatigues in fingers or hands of a physician operating the handle device. He or she can concentrate for precision of the manual operation.

Various methods are available for connecting the metal tube 51. In FIG. 14B, the spiral groove 52 is formed in the metal tube 51. End turns of the helical coil 50 are fitted in and wound on the spiral groove 52 before wax is used for attachment. Alternatively, ends of the metal tube 51 and the helical coil 50 can be welded together for attachment. Examples of the metal tube 51 are stainless steel pipe, nickel-titanium alloy pipe or the like, with an inner diameter of 0.6 mm and an outer diameter of 1 mm.

### EXAMPLE 7

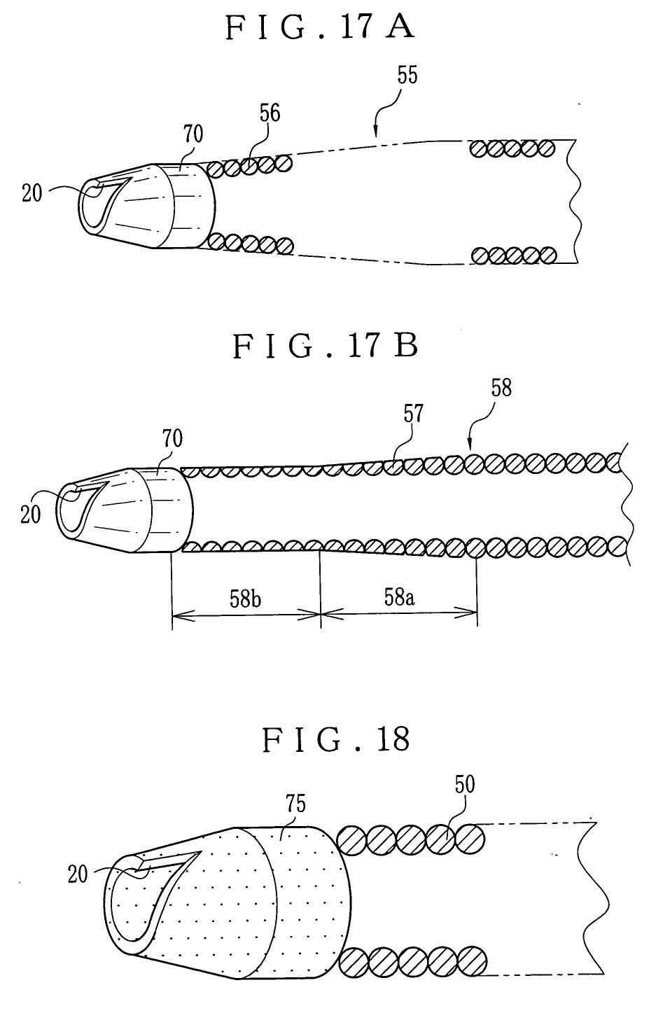

In FIGS. 17A and 17B, tubular windings or helical coils of Example 7 are illustrated, the structures including a first structure of FIG. 17A, and a second structure of FIG. 17B. In a tubular winding or helical coil 55, the above-described examples are repeated with an exception in which at least a portion of the helical coil 55 has a decreasing diameter toward the distal end. This shape raises rotational force transmitted to the distal end. In FIG. 17A, the helical coil 55 has a wire 56, and constituted by winding the wire 56 with an inclination to decrease the diameter. In FIG. 17B, a tubular winding or helical coil 58 has a wire 57. A radius-decreasing portion 58a is formed by winding the wire 57 cylindrically and then by cutting the outside of the wire 57 with an inclination from the proximal end toward the distal end. The helical coil 58 has an inner diameter constant unlike the outer diameter. The inclination can be small enough. In the helical coil 58, determining the very small outer diameter is ensured. In FIG. 17A, the diameter of the helical coil 55 decreases from 1 mm to 0.8 mm from the proximal end to the distal end. The diameter of the end face of the tubular tip 70 is 0.6 mm. The torque or twisting moment is determined according to a ratio of radii of rotational elements. Thus, the torque at the distal end can be raised at approximately 1.25 times. The torque at the end face can be raised at approximately 1.66 times. In FIG. 17B, an equiradial portion 58b is formed in the helical coil 58, so that flexibility of a distal end can be ensured.

### EXAMPLE 8

In FIG. 18, a hollow tip is illustrated. Each one of medical instruments of Examples 1-7 is repeated, but in which a tubular tip 75 is formed from radiopaque material. To this end, various methods can be used, for example a method of preparing a separate radiopaque part, and securing the tubular tip 75 on the tubular winding or helical coil 50, a method of securing a separate tip to the helical coil 50 after plating the tip with radiopaque material, a method of forming a cutting head from radiopaque wax to constitute the tubular tip 75, and the like. It is possible to recognize an end position of a cutting head easily under radioactive radiation for treating a lesion. Examples of radiopaque examples are gold, platinum, tungsten and the like.

### EXAMPLE 9

One preferred method of the invention is production of the rotational medical instrument 100 having a tubular shape in which the guide wire 170 is insertable, and including the helical coil 50 having a predetermined length and the tubular tip 60 secured to a distal end of the helical coil 50. The method includes first and second steps, the first step being forming the helical coil 50 by winding or twisting wire or strands of metal. The second step is securing the tubular tip 60 to the distal end of the helical coil 50 after forming the cutting head 10 on the tubular tip 60.

Another preferred method of the invention is production of the rotational medical instrument 100 having a tubular shape in which the guide wire 170 is insertable, and including the helical coil 50 having a predetermined length and the tubular tip 60 secured to a distal end of the helical coil 50. The method includes first and second steps, the first step being forming the helical coil 50 by winding or twisting wire or strands of metal. The second step is forming the cutting head 10 on the tubular tip 60 after securing the tubular tip 60 to the distal end of the helical coil 50.

### EXAMPLE 10

One preferred method of the invention is production of the rotational medical instrument 100 having a tubular shape in which the guide wire 170 is insertable, and including the helical coil 50 having a predetermined length and the tubular tip 60 secured to a distal end of the helical coil 50. The method includes first and second steps, the first step being forming the helical coil 50 by winding or twisting wire or strands of metal. The second step is securing the tubular tip 60 to the distal end of the helical coil 50 after forming the cutting head 10 on the tubular tip 60, the cutting head 10 being so directed that a rotational direction of cutting of the tubular tip 60 is the same as a rotational direction of reducing a diameter of the helical coil 50 by winding.

Another preferred method of the invention is production of the rotational medical instrument 100 having a tubular shape in which the guide wire 170 is insertable, and including the helical coil 50 having a predetermined length and the tubular tip 60 secured to a distal end of the helical coil 50. The method includes first and second steps, the first step being forming the helical coil 50 by winding or twisting wire or strands of metal. The second step is forming the cutting head 10 on the tubular tip 60 after securing the tubular tip 60 to the distal end of the helical coil 50, the cutting head 10 being so directed that a rotational direction for cutting of the tubular tip 60 is the same as a rotational direction of reducing a diameter of the helical coil 50 by winding.

### EXAMPLE 11

A combination is constituted by any one of examples of the rotational medical instrument 100 and the guide wire 170 described heretofore. The combination has the helical coil 50 and the tubular tip 60 secured to the end of the helical coil 50 in a range having a predetermined length in the axial direction. The rotational medical instrument 100 of which the tubular tip 60 has the cutting head 10 is disposed about the guide wire 170 which includes the helical coil 180 and the wire 171 inserted in the helical coil 180, for use in treating a lesion.

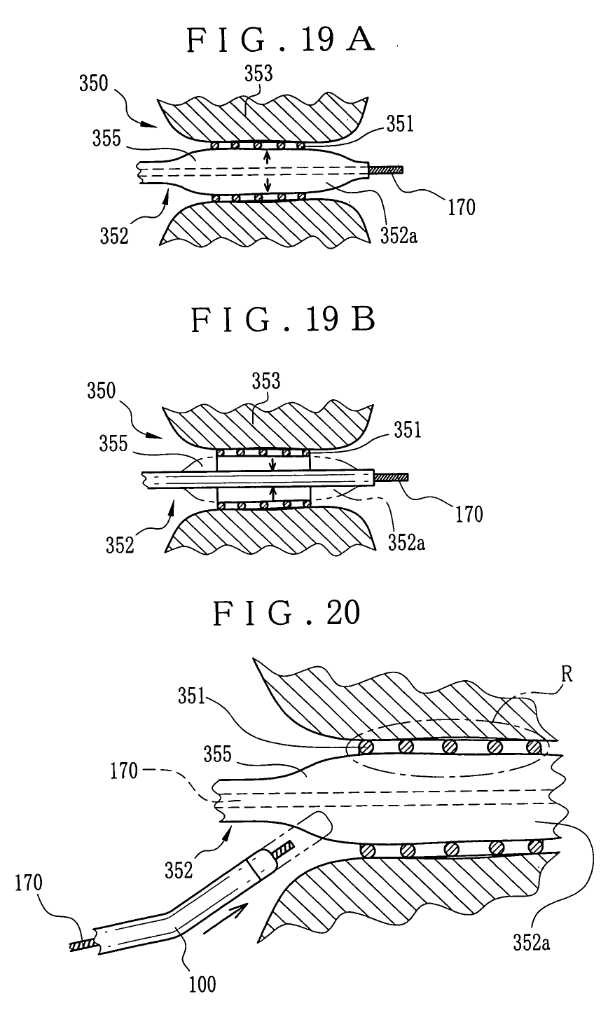

In FIGS. 19A and 19B, the use of the rotational medical instrument 100 of the invention is illustrated, in which a balloon catheter at a lesion is compressed and removed. In FIG. 20, a state of perforating of a balloon catheter is illustrated. A balloon catheter, even when it cannot be removed from a human lumen in a well-known method, can be removed easily by use of a combination of the rotational medical instrument 100 and the guide wire 170 of any one of above examples.

For a body of a patient, a lesion 350 of FIG. 19A is penetrated by the guide wire 170. A balloon catheter 352 is provided with a stent 351, as a piece of a pipe with numerous openings in a meshed shape. A constriction 353 is given a larger space by deployment of the stent 351. The stent 351 is kept internally for treatment by enlarging a diameter. In FIG. 19B, a compressed state of a balloon 352a is illustrated. Note that a drug eluting type of the stent 351 has been known recently in the field of medicine for treatment with the stent 351. In the region R in FIG. 20, the stent 351 is accidentally stuck on the balloon 352a, and may stable without possibility to remove the balloon 352a. Failure may occur in compressing the balloon 352a due to the deployed form of the stent 351 upon adhesion or sticking of the stent 351. Also, physiological saline water 355 is used to enlarge or compress the balloon 352a. If compression is impossible, surgical operation will be required due to failure in removal of the balloon 352a. This being so, a physician must treat the patient immediately in view of the body condition of the patient.

For this situation, the rotational medical instrument 100 of FIG. 20 is introduced to the vicinity of the balloon 352a of the balloon catheter 352 while guided by the guide wire 170. The rotational medical instrument 100 is caused to project and rotate on the guide wire 170, to perforate the balloon 352a for shrinkage in a forced manner. Therefore, the balloon catheter 352 can be withdrawn. The tubular tip 60 according to the in invention can thrust in and perforate the balloon in a reliable manner, because the cutting head 10 of the tubular tip 60 has a sharp end portion and a cutting blade portion.

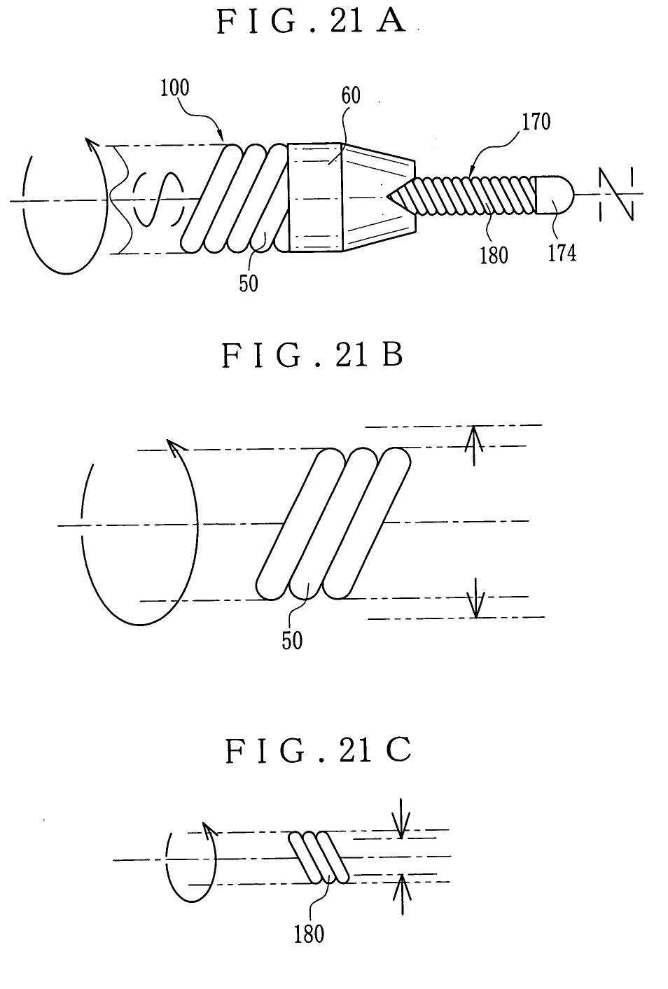

The following is effects of the feature in that a rotational direction of the helical coil 50 of the cutting head 10 is opposite to that of the helical coil 180 of the guide wire 170. See FIGS. 21A-21C. The advance of the rotational medical instrument 100 with the guide wire 170 into a lesion is facilitated at the same time as the cutting by rotation of the rotational medical instrument 100 is facilitated.

In FIGS. 21A-21C, directions of turns in the helical coil 50 are illustrated. If the direction of turns in the rotational medical instrument 100 is the S direction or a left screw direction, the helical coil 50 is moved forwards into an occlusion when rotated in the counterclockwise direction as viewed from the proximal end. The guide wire 170 in the rotational medical instrument 100 is likely to rotate together with the helical coil 50. If the direction of turns in the guide wire 170 in the helical coil 180 is the Z direction or a right screw direction, the helical coil 180 comes to have an decreasing diameter by twisting. This is illustrated in FIG. 21C. There is interaction between operation increasing the diameter of the helical coil 50 (FIG. 21B) and operation decreasing the diameter of the helical coil 180 of the guide wire 170 (FIG. 21C) in response to the rotation of the rotational medical instrument 100 in the counterclockwise direction as viewed from the proximal end. This is effective in keeping a clearance between the guide wire 170 and the rotational medical instrument 100. The handling of those can be easy and also accurate even with their small diameters.

### EXAMPLE 12

The rotational medical instrument 100 for cutting by rotation is likely to cause a problem in delay in rotation between proximal and distal ends. If excessive torque is created at the proximal end by manual operation, perforation of a blood vessel may be caused in the transmission of rotation with the delay. In the invention, the over torque preventing mechanism is disposed for creating slipped rotation upon excess of the torque, to avoid perforation of a blood vessel due to transmitted excessive torque to the distal end. In particular, the force is transmitted to the distal end after occurrence of a twist. Lag in the rotation is likely to occur between the proximal and distal ends, to cause the above-described failure. However, the over torque preventing mechanism makes it possible for a physician to treat a patient safely and reliably.

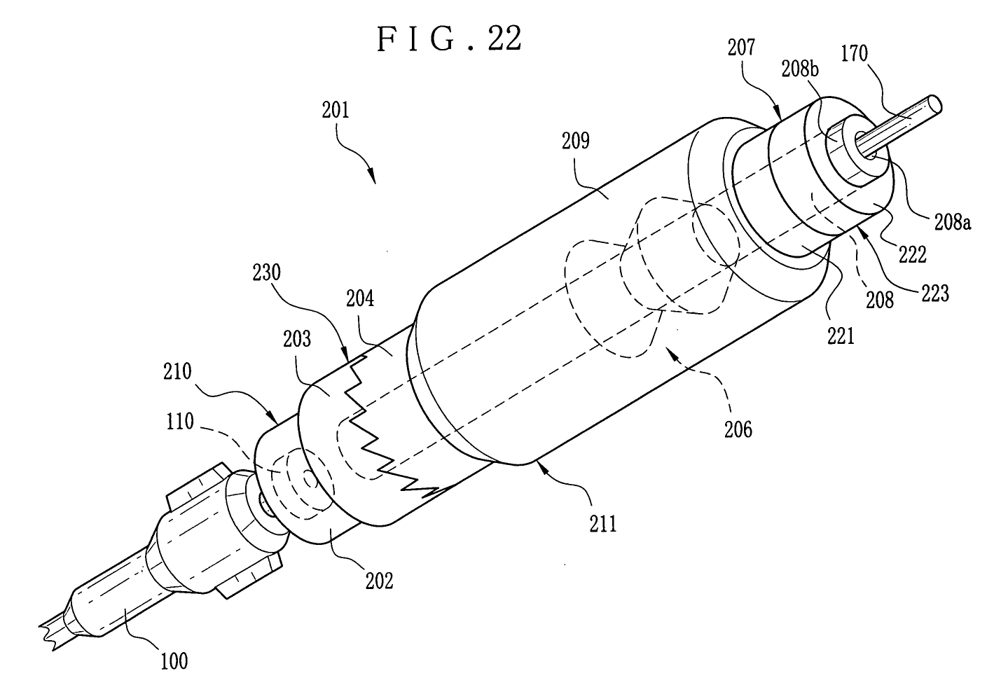

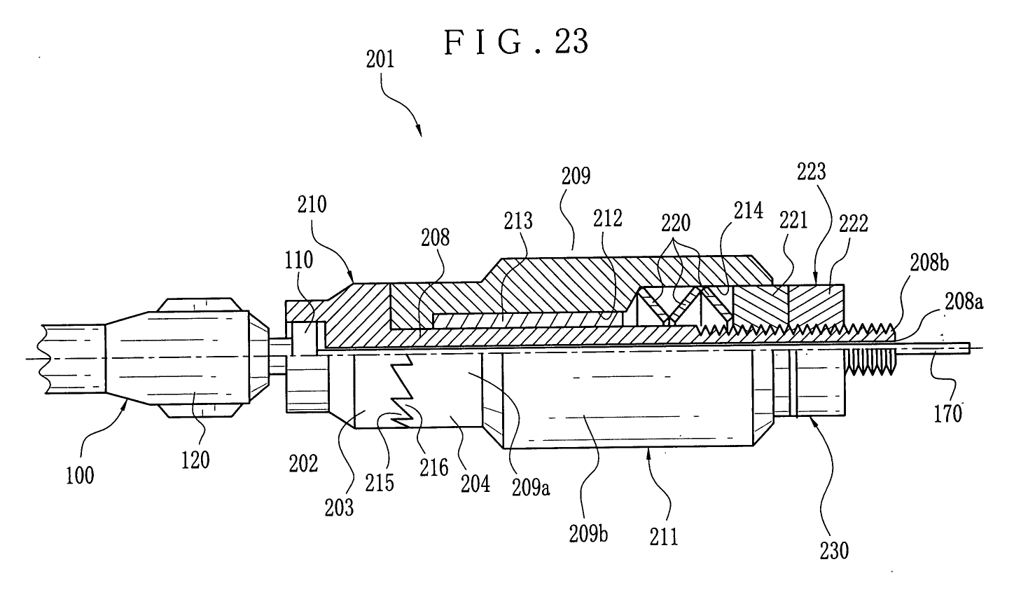

In FIG. 22, a preferred rotational handle device 201 for atherectomy is illustrated. In FIG. 23, the same is depicted in a section. An over torque preventing mechanism for torque limiting is typically incorporated. In FIGS. 22 and 23, the rotational handle device 201 includes a Luer lock portion 202, a gear mechanism, a spring 206, an adjusting thread portion 207, a shaft or inner sheath 208, and a handle housing 209 in a rotational handle device. The Luer lock portion 202 is connectable with a proximal portion of the rotational medical instrument 100. The gear mechanism includes a first face gear 203 and a second face gear 204. The spring 206 biases for mesh between the first and second face gears 203 and 204. The adjusting thread portion 207 adjusts biasing of the spring 206. The shaft 208 is connected with the adjusting thread portion 207. A wire lumen 208a is formed through the shaft 208 for inserting the guide wire 170.

In FIG. 23, a first rotatable tube 210 is a composite element having the Luer lock portion 202, the first face gear 203 and the shaft or inner sheath 208. The Luer lock portion 202 is at the end of the first rotatable tube 210. The Luer or screw 110 of the rotational medical instrument 100 is helically coupled with and fastened on the Luer lock portion 202. The shaft 208 has a smaller diameter, and formed with a rear end of the Luer lock portion 202 on the right side as viewed in the drawing. A male thread portion 208b is formed on the rear end of the shaft 208 for fitting of a lock nut of the adjusting thread portion 207. Also, the wire lumen 208a is formed through the shaft 208 for guide wire insertion.

The first face gear 203 is formed with a rear end of the Luer lock portion 202. Gear teeth or projections 215 are included in the first face gear 203, and extend radially by way of regularly arranged ridges. Each of the gear teeth 215 is triangular when viewed in section.

A second rotatable tube 211 is a composite part having the second face gear 204 and the handle housing 209. The second rotatable tube 211 receives insertion of the shaft or inner sheath 208 of the first rotatable tube 210. The second rotatable tube 211 includes the second face gear 204 for mesh with the first face gear 203. Gear teeth or projections 216 are included in the second face gear 204 and have a similar shape as that of the gear teeth 215 of the first face gear 203. A lumen 212 is formed in the second rotatable tube 211 axially to come through. A sleeve 213 of a synthetic resin keeps the shaft 208 rotatable on the lumen 212. A spring chamber 214 is formed to extend from the rear of the lumen 212. Note that a bearing may be used in place of or in addition to the sleeve 213.

Three panel shaped biasing springs 220 are contained in the spring chamber 214 and directed alternately with one another. The panel shaped springs 220 bias the second rotatable tube 211 toward the first rotatable tube 210. An adjusting nut or fitting nut 221 and a locking nut 222 are helically fastened on the male thread portion 208b of the shaft or inner sheath 208. A tube support 223 is constituted by the adjusting nut 221, the locking nut 222 and the shaft 208, and keeps the second rotatable tube 211 rotatable on the first rotatable tube 210. Also, the face gears 203 and 204 are kept movable by the tube support 223 and the panel shaped springs 220 between a first position for mesh with one another and a second position where the second face gear 204 is free from the first face gear 203 to rotate with slip. Thus, a small torque equal to or smaller than predetermined level is transmitted from the second rotatable tube 211 to the first rotatable tube 210 by the mesh of the first face gear 203 with the second face gear 204. Also, a great torque greater than the predetermined level is prevented from transmission, because the second rotatable tube 211 rotates freely upon disengagement of the second face gear 204 from the first face gear 203. In short, an over torque preventing mechanism 230 for torque limiting is a composite unit including the first face gear 203, the second face gear 204, the panel shaped springs 220 and the tube support 223, the tube support 223 having the shaft 208, the adjusting nut 221 and the locking nut 222.

The bias of the panel shaped biasing springs 220 can be adjusted by changing the position of the adjusting nut 221 at the shaft or inner sheath 208. The torque for transmission can be changed.

The second rotatable tube 211 is constituted by a small diameter sleeve 209a and a large diameter sleeve 209b. The small diameter sleeve 209a has an outer diameter equal to that of the first rotatable tube 210.

The operation of the rotational handle device 201 for the medical instrument of the invention is illustrated. At first, the guide wire 170 is inserted in a blood vessel. Then the rotational medical instrument 100 is inserted by following the surface of the guide wire 170. The first rotatable tube 210 of the rotational handle device 201 is connected with the rotational medical instrument 100. The second rotatable tube 211 is rotated by manually holding either of the small diameter sleeve 209a and the large diameter sleeve 209b. Rotation of the second rotatable tube 211 is transmitted by the over torque preventing mechanism 230 to the first rotatable tube 210, to rotate the rotational medical instrument 100. If a torque applied to the second rotatable tube 211 is more than a predetermined level, the second face gear 204 is disengaged from the first face gear 203. So the second face gear 204 rotates freely upon slip between the gear teeth 215 and 216. The distal end of the rotational medical instrument 100 is prevented from application of excessively high torque.

In FIG. 22, the panel shaped springs 220 are three, which is advantageous in great load and easy change ability in the spring constant. However, one or more compression coil spring may be used instead. The handle housing 209 of the second rotatable tube 211 has the small diameter sleeve 209a and the large diameter sleeve 209b for changing over rotation in a light state and a heavy state. The small diameter sleeve 209a can be selected if necessary. However, the handle housing 209 may have a single diameter, and also may have three or more diameters in a composite shape. Also, the rotational handle device 201 can be directly in connection with a proximal side of the rotational medical instrument 100 in place of the use of the connector 120 for connection with the Luer lock portion 202.

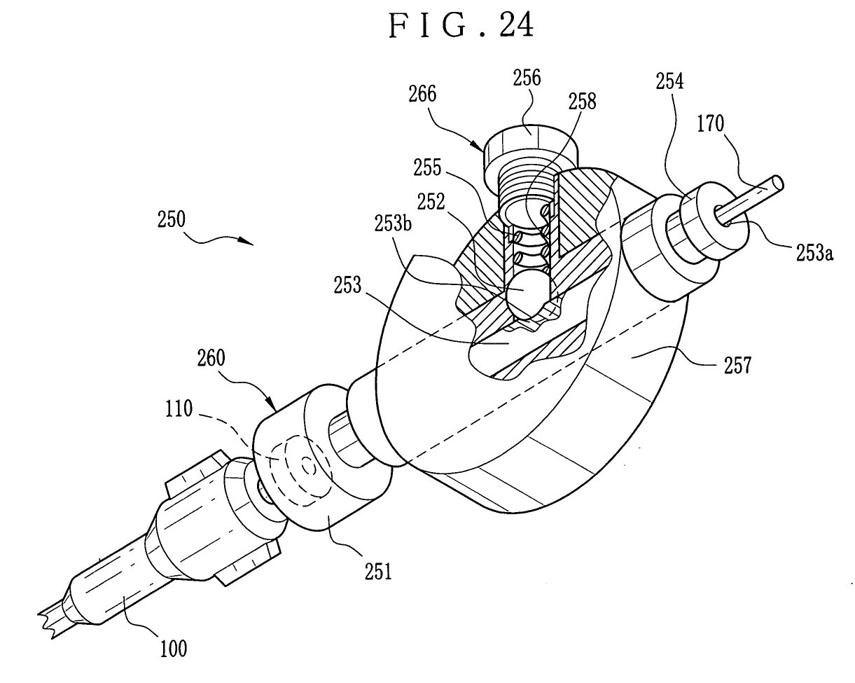

In FIG. 24, another preferred handle device is illustrated. This is characterized in the use of a lock ball. In FIG. 24, a rotational handle device 250 includes a Luer locking portion 251, a shaft or inner sheath 253, and a holding second rotatable tube 257. The Luer locking portion 251 is connectable with a proximal end of the rotational medical instrument 100. A lock ball 252 is movable and engageable in a recess in the shaft 253. A compression coil spring 255 for biasing biases the lock ball 252. An adjusting screw 256 adjusts a space for the compression coil spring 255 to determine the bias. The holding second rotatable tube 257 contains the lock ball 252 and the adjusting screw 256. A lumen 253a is formed through the shaft 253 for insertion of the guide wire 170. A nut 254 at the end of the shaft 253 keeps the holding second rotatable tube 257 secured to the shaft 253. A ball chamber 258 is formed in the holding second rotatable tube 257 and contains the lock ball 252.

A first rotatable tube 260 is constituted by the Luer locking portion 251 and the shaft or inner sheath 253. The holding second rotatable tube 257 is combined with the first rotatable tube 260. A shaft recess 253b is formed in the shaft 253. An over torque preventing mechanism 266 for torque limiting is constituted by the shaft recess 253b, the lock ball 252, the compression coil spring 255, and the adjusting screw 256. Note that the holding second rotatable tube 257 is the thick panel in an elliptical shape, but may be a panel of an oval shape, or other shape of a grip form.

The compression coil spring 255 is adjusted by the adjusting screw 256 for predetermined force of compression. When the holding second rotatable tube 257 rotates, the shaft or inner sheath 253 rotates together if the torque of the holding second rotatable tube 257 is equal to or lower than a predetermined level. In contrast, if the torque of the holding second rotatable tube 257 is equal to or higher than the predetermined level, the lock ball 252 becomes offset from the shaft recess 253b in the shaft 253, to cause rotational slip of the holding second rotatable tube 257. This prevents transmission of excessive torque to the distal end of the rotational medical instrument 100. Note that one or more single panel shaped springs may be used in place of the compression coil spring 255 in FIG. 24. Also, if the compression coil spring 255 has such a characteristic that its spring constant is stably unchanged, the adjusting screw 256 may not be used. A screw for setting the compression coil spring 255 without adjustment can be used in place of the adjusting screw 256. Also, the Luer locking portion 251 may be eliminated. The rotational handle device 250 can be connected to a proximal side of the rotational medical instrument 100.

Torque is transmitted by retention in the above embodiment. However, friction is used to transmit torque. In FIG. 23, flat surfaces are formed in place of the face gears 203 and 204. A friction sheet is set between the flat surfaces. When a great torque greater than the predetermined level is applied to the holding second rotatable tube 257, the holding second rotatable tube 257 rotates in a slipped manner. No further transmission of torque occurs between the first and second rotatable tubes 260 and 257.

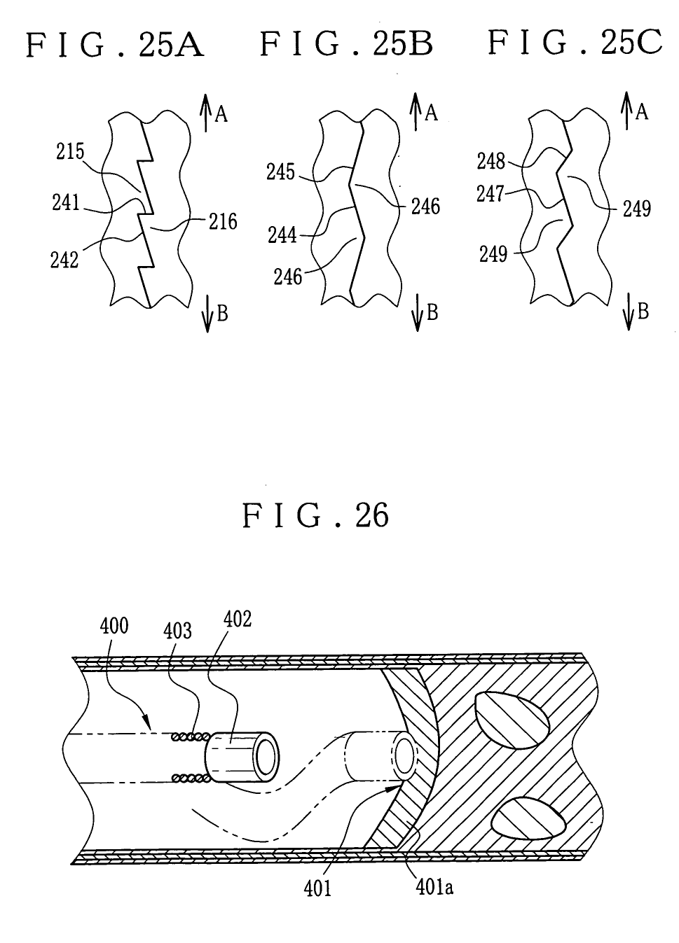

In FIG. 23, the face gears 203 and 204 are depicted with an exaggeration to clarify their meshed state. Heights of the gear teeth 215 and 216 are not limited to that being depicted, because determined according to toque for transmission. In FIGS. 25A-25C, shapes of gear teeth are illustrated. In FIG. 25A, the gear teeth 215 and 216 have inclined tooth surfaces 241 and 242. In FIG. 25B, gear teeth or projections 246 of a face gear have inclined tooth surfaces 244 and 245, which are arranged in an isosceles triangle, and between which an obtuse angle is defined. In FIG. 25C, gear teeth or projections 249 of a face gear have inclined tooth surfaces 247 and 248, which are different in the angle of inclination.

In the gear teeth 215 and 216 of FIG. 25A, the inclined tooth surface 241 comes in mesh with one another upon rotation in the direction A so that normal transmission of rotation is enabled without prevention of over torque. In contrast, inclined surfaces become engaged with one another upon rotation in the direction B, so that over torque prevention is effected. This is a clutch mechanism in which transmission is changed over between the prevention of over torque and a normal mode without prevention of over torque. In FIG. 25B, prevention of over torque occurs in both of the directions A and B owing to the inclined tooth surfaces 244 and 245 defining an isosceles triangle. In FIG. 25C, the transmitted torque is changed over for high and low values between the two directions A and B owing to the inclined tooth surfaces 247 and 248.

The combined use with the rotational handle device 201 or 250 has been described heretofore. The following is effects and other features of the use of the rotational medical instrument 100.

At first, a rotational direction for cutting of the cutting head of the tubular tip 60 is set equal to a rotational direction to reduce the diameter of winding of the helical coil 50, to obtain higher ability of cutting. This is because the helical coil 50 can become rigid in a temporary manner when rotated, to heighten ability of transmission of the tubular tip 60 for torque. This is preferable if a size of the lesion is relatively short, and if the lesion is relatively hard.

In contrast, if soft and hard materials exist in mixture and with a relatively great length of a lesion, it is preferable to set a rotational direction of cutting of a cutting blade portion equal to a rotation direction of increasing a diameter of the helical coil 50. This is because the helical coil 50 advances in the forward direction when rotated, and also increases its diameter. Rotational force of cutting is transmitted in a direction toward the cutting head 10 of the tubular tip 60. If the helical coil 50 is rotated in reverse, the helical coil 50 decreases its diameter, and is easy to exit from the long lesion. It is to be noted that each one of the two combinations of the rotational directions can be selected by considering forms of lesions to be treated.

Note that a tube of resin can be additionally inserted in the rotational medical instrument 100 for further smoothing contact with the guide wire 170. Also, wire in the helical coil 50 for the rotational medical instrument 100 may be wire coated with resin. Furthermore, an outer portion of the helical coil 50 may be coated with thin film of resin, for various purposes of lowering sticking of thrombus, preventing leakage of fluid between turns of the helical coil 50, and the like. In the above examples, the hollow tube of metal is fixedly secured to a proximal end of the rotational medical instrument 100. Also, a medical tube assembly containing a tube of resin may be used. A thin wire of metal may be knitted or combined about the tube of resin, and then be coated with resin of a coating, as medical tube assembly.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A medical instrument for treatment in a tubular form including a tube for insertion by guiding of a medical guide wire inside, said medical instrument comprising:
said tube including:
a helical coil of metal;
a tubular tip of metal secured to an end of said helical coil; and
a cutting head of a blade shape, formed on said tubular tip, for cutting a lesion upon being rotated.

2. A medical instrument for treatment as defined in claim 1, wherein said tubular tip has a shape with a decreasing diameter toward a distal end thereof.

3. A medical instrument for treatment as defined in claim 2, wherein said tubular tip includes a U shaped cut or V shaped notch, formed in said cutting head at a front face and an outer face thereof, and directed in a direction along a tubular tip axis.

4. A medical instrument for treatment as defined in claim 2, wherein said cutting head includes a cut, formed to retreat from a front end face thereof, and having a cut surface inclined with reference to an axis of said tubular tip.

5. A medical instrument for treatment as defined in claim 2, wherein said tubular tip includes a spiral groove formed in a tubular tip outer surface to extend helically from a front end face of said cutting head.

6. A medical instrument for treatment as defined in claim 2, wherein said helical coil is a multi filament helical coil constituted by helically wound plural wires of metal.

7. A medical instrument for treatment as defined in claim 2, wherein said helical coil is shaped by helically winding a wire after twisting plural strands of metal in a form of said wire.

8. A medical instrument for treatment as defined in claim 2, wherein said helical coil is so wound as to decrease a diameter upon being rotated in a first rotational direction, and causes said cutting head to cut said lesion.

9. A medical instrument for treatment as defined in claim 2, wherein said helical coil has a shape with a decreasing diameter toward a distal end thereof.

10. A medical instrument for treatment as defined in claim 2, wherein said tubular tip is radiopaque.

11. A rotational handle device for rotating a medical instrument for treatment in a tubular form including a tube for insertion by guiding of a medical guide wire inside, said rotational handle device comprising:
wherein said tube includes a helical coil of metal, a tubular tip of metal secured to an end of said helical coil, and a cutting head of a blade shape, formed on said tubular tip, for cutting a lesion upon being rotated;
a first rotatable tube for rotating with said tube, said first rotatable tube having a wire lumen for receiving insertion of said medical guide wire;
an externally operable second rotatable tube secured to said first rotatable tube in an axial direction;
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube.

12. A rotational handle device as defined in claim 11, wherein one of said first and second rotatable tubes has a retaining projection, and a remaining one thereof has a retaining recess engageable with said retaining projection;
said over torque preventing mechanism includes:
a tube support for keeping said first and second rotatable tubes movable in said axial direction between first and second positions, wherein said retaining projection retains said retaining recess when said first and second rotatable tubes are in said first position, and releases said retaining recess when said first and second rotatable tubes are in said second position;
a biasing portion for biasing said first or second rotatable tube with a predetermined bias, for retaining said first and second rotatable tubes in said first position upon application of said torque equal to or lower than said predetermined level, to rotate said first and second rotatable tubes together, and for setting said first and second rotatable tubes in said second position upon application of said torque higher than said predetermined level, to rotate said second rotatable tube free from said first rotatable tube.

13. A rotational handle device as defined in claim 12, wherein said retaining projection is a plurality of first ridges formed on a tube end face of said first rotatable tube to extend radially;
said second rotatable tube includes a plurality of second ridges formed on a tube end face thereof to extend radially, to define said retaining recess between, for mesh with said first ridges when said first and second rotatable tubes are in said first position, and for disengagement from said first ridges when said first and second rotatable tubes are in said second position.

14. A rotational handle device as defined in claim 13, wherein said first and second ridges are teeth, and are in a triangular shape as viewed in a section perpendicular to a direction of extending radially.

15. A rotational handle device as defined in claim 11, wherein said over torque limiting mechanism includes:
a first gear formed on said first rotatable tube;
a second gear, formed on said second rotatable tube, for mesh with said first gear;
a biasing portion for pressing said second gear on said first gear;
wherein said second gear is rotatable free from said first gear when said torque applied to said first and second gears becomes higher than said predetermined level.

16. A rotational handle device as defined in claim 11, wherein said first rotatable tube includes a shaft for supporting said second rotatable tube in a rotatable manner;
said over torque preventing mechanism includes:
a shaft recess formed in said shaft;
a lock ball engageable with said shaft recess;
a ball chamber for containing said lock ball movably between a first position where said lock ball is shifted in said shaft recess and a second position where said lock ball is offset from said shaft recess;
a biasing portion for biasing said lock ball with a predetermined bias, for retaining said lock ball in said first position upon application of said torque equal to or lower than said predetermined level, to rotate said first and second rotatable tubes together, and for setting said lock ball in said second position upon application of said torque higher than said predetermined level, to rotate said second rotatable tube free from said first rotatable tube.

17. A rotational handle device as defined in claim 16, further comprising an adjuster for adjusting said biasing portion in relation to said bias.

18. A rotational handle device as defined in claim 11, wherein said second rotatable tube includes a first sleeve and a second sleeve having a smaller diameter than said first sleeve.

19. A medical equipment for treatment, comprising:
a medical instrument for treatment in a tubular form including a tube for insertion by guiding of a medical guide wire inside;
wherein said tube includes a helical coil of metal, a tubular tip of metal secured to an end of said helical coil, and a cutting head of a blade shape, formed on said tubular tip, for cutting a lesion upon being rotated;
a first rotatable tube for rotating with said tube, said first rotatable tube having a wire lumen for receiving insertion of said medical guide wire;
a connector for removably connecting said tube with said first rotatable tube;
an externally operable second rotatable tube, secured to said first rotatable tube in an axial direction, for rotationally operating said medical instrument on a proximal side; and
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube.

20. A guide wire fitted medical equipment for treatment, comprising:
a medical instrument for treatment in a tubular form including a tube;
wherein said tube includes a helical coil of metal, a tubular tip of metal secured to an end of said helical coil, and a cutting head of a blade shape, formed on said tubular tip, for cutting a lesion upon being rotated;
a first rotatable tube for rotating with said tube, said first rotatable tube having a wire lumen;
a connector for removably connecting said tube with said first rotatable tube;
an externally operable second rotatable tube, secured to said first rotatable tube in an axial direction, for rotationally operating said medical instrument on a proximal side;
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube;
a medical guide wire, having a helical coil on at least a distal end thereof, for guiding insertion of said tube and said first rotatable tube thereabout;
wherein a direction of winding of said helical coil of said medical instrument is reverse to a direction of winding of said helical coil of said medical guide wire.

21. A rotational handle device for rotationally operating a medical instrument for treatment on a proximal side, said medical instrument in a tubular form including a tube for insertion by guiding of a medical guide wire inside, wherein said tube includes a helical coil of metal, and a tip secured to an end of said helical coil, said rotational handle device comprising:
a first rotatable tube for rotating with said tube, said first rotatable tube having a wire lumen for receiving insertion of said medical guide wire;
an externally operable second rotatable tube secured to said first rotatable tube in an axial direction;
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube.

22. A rotational handle device as defined in claim 21, wherein one of said first and second rotatable tubes has a retaining projection, and a remaining one thereof has a retaining recess engageable with said retaining projection;
said over torque preventing mechanism includes:
a tube support for keeping said first and second rotatable tubes movable in said axial direction between first and second positions, wherein said retaining projection retains said retaining recess when said first and second rotatable tubes are in said first position, and releases said retaining recess when said first and second rotatable tubes are in said second position;
a biasing portion for biasing said first or second rotatable tube with a predetermined bias, for retaining said first and second rotatable tubes in said first position upon application of said torque equal to or lower than said predetermined level, to rotate said first and second rotatable tubes together, and for setting said first and second rotatable tubes in said second position upon application of said torque higher than said predetermined level, to rotate said second rotatable tube free from said first rotatable tube.

23. A rotational handle device as defined in claim 22, wherein said retaining projection is a plurality of first ridges formed on a tube end face of said first rotatable tube to extend radially;
said second rotatable tube includes a plurality of second ridges formed on a tube end face thereof to extend radially, to define said retaining recess between, for mesh with said first ridges when said first and second rotatable tubes are in said first position, and for disengagement from said first ridges when said first and second rotatable tubes are in said second position.

24. A rotational handle device as defined in claim 23, wherein said first and second ridges are teeth, and are in a triangular shape as viewed in a section perpendicular to a direction of extending radially.

25. A rotational handle device as defined in claim 21, wherein said over torque limiting mechanism includes:
a first gear formed on said first rotatable tube;
a second gear, formed on said second rotatable tube, for mesh with said first gear;
a biasing portion for pressing said second gear on said first gear;
wherein said second gear is rotatable free from said first gear when said torque applied to said first and second gears becomes higher than said predetermined level.

26. A rotational handle device as defined in claim 21, wherein said first rotatable tube includes a shaft for supporting said second rotatable tube in a rotatable manner;
said over torque preventing mechanism includes:
a shaft recess formed in said shaft;
a lock ball engageable with said shaft recess;
a ball chamber for containing said lock ball movably between a first position where said lock ball is shifted in said shaft recess and a second position where said lock ball is offset from said shaft recess;
a biasing portion for biasing said lock ball with a predetermined bias, for retaining said lock ball in said first position upon application of said torque equal to or lower than said predetermined level, to rotate said first and second rotatable tubes together, and for setting said lock ball in said second position upon application of said torque higher than said predetermined level, to rotate said second rotatable tube free from said first rotatable tube.

27. A rotational handle device as defined in claim 26, further comprising an adjuster for adjusting said biasing portion in relation to said bias.

28. A rotational handle device as defined in claim 21, wherein said second rotatable tube includes a first sleeve and a second sleeve having a smaller diameter than said first sleeve.

29. A medical equipment for treatment, comprising:
a medical instrument for treatment in a tubular form including a tube for insertion by guiding of a medical guide wire inside, wherein said tube includes a helical coil of metal, and a tip secured to an end of said helical coil;
a first rotatable tube for rotating with said tube, said first rotatable tube having a wire lumen for receiving insertion of said medical guide wire;
a connector for removably connecting said tube with said first rotatable tube;
an externally operable second rotatable tube, secured to said first rotatable tube in an axial direction, for rotationally operating said medical instrument on a proximal side; and
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube.

30. A guide wire fitted medical equipment for treatment, comprising:
a medical instrument for treatment in a tubular form including a tube, wherein said tube includes a helical coil of metal, and a tip secured to an end of said helical coil;
a first rotatable tube for rotating with said tube, said first rotatable tube having a wire lumen;
a connector for removably connecting said tube with said first rotatable tube;
an externally operable second rotatable tube, secured to said first rotatable tube in an axial direction, for rotationally operating said medical instrument on a proximal side;
an over torque preventing mechanism, disposed between said first and second rotatable tubes, for transmitting rotation of said second rotatable tube to said first rotatable tube upon application of torque equal to or lower than a predetermined level to said second rotatable tube, and for causing said second rotatable tube to rotate free from said first rotatable tube upon application of torque higher than said predetermined level to said second rotatable tube;
a medical guide wire, having a helical coil on at least a distal end thereof, for guiding insertion of said tube and said first rotatable tube thereabout;
wherein a direction of winding of said helical coil of said medical instrument is reverse to a direction of winding of said helical coil of said medical guide wire.
